(19) United States
(12) Patent Application Publication
(10) Pub. No.: US 2023/0381399 A1
(43) Pub. Date: Nov. 30, 2023

(54) CATHETER TIP

(71) Applicant: Auris Health, Inc., Redwood City, CA (US)

(72) Inventors: Russell W. PONG, Newark, CA (US); Thomas R. JENKINS, Alameda, CA (US); Ka Chun WONG, South San Francisco, CA (US)

(21) Appl. No.: 18/324,912

(22) Filed: May 26, 2023

Related U.S. Application Data
(63) Continuation of application No. PCT/IB2021/062089, filed on Dec. 21, 2021.
(60) Provisional application No. 63/132,885, filed on Dec. 31, 2020.

(51) Int. Cl. A61M 1/00; A61M 25/01; A61M 25/00

(52) U.S. Cl. CPC A61M 1/774; A61M 25/0147; A61M 25/001

(57) ABSTRACT

An aspiration catheter can include an elongate shaft and an instrument base coupled to the shaft and configured to control actuation of at least a distal portion of the shaft. The shaft can include a lumen configured to couple to an aspiration system to provide aspiration to a target site, such as to remove an object from a patient. At least a portion of the distal end portion of the shaft can include an inner diameter that is smaller than an inner diameter of the rest of shaft to prevent objects that are larger than a particular size from entering.

### RELATED APPLICATION(S)

This application is a continuation of International Application No. PCT/IB2021/062089, filed Dec. 21, 2021, and entitled CATHETER TIP, which claims the benefit of priority to U.S. Provisional Application No. 63/132,885, filed Dec. 31, 2020, and entitled CATHETER TIP, the disclosures of which are hereby incorporated by reference in their entirety.

### BACKGROUND

Various medical procedures involve the use of one or more medical devices for accessing a target anatomical site in a patient. In some instances, the improper use of certain devices when accessing the site in connection with a procedure can adversely affect the health of the patient, the integrity of the medical device(s), and/or the efficacy of the procedure.

### SUMMARY

In some implementations, the present disclosure relates to a catheter comprising an elongate shaft and an instrument base coupled to the elongate shaft and configured to control actuation of the elongate shaft. The elongate shaft includes a distal section, a middle section, a proximal section, and a lumen. The middle section includes a first inner diameter and at least a portion of the distal section includes a second inner diameter that is smaller than the first inner diameter. The lumen is configured to couple to an aspiration system to provide aspiration to a target site via the lumen.

In some embodiments, a ratio of the second inner diameter to the first inner diameter is within a range of 0.5 to 0.9. Further, in some embodiments, a longitudinal length of the at least the portion of the distal section that includes the second inner diameter is less than the second inner diameter.

In some embodiments, the catheter further comprises an elongate movement member coupled to the distal section of the elongate shaft. The instrument base can be configured to manipulate the elongate movement member to control actuation of the elongate shaft.

In some embodiments, the distal section of the elongate shaft includes a first portion and a second portion that is distal to the first portion. The first portion can include the second inner diameter. The second portion can include a third inner diameter that is larger than the second inner diameter. In examples, a longitudinal length of the first portion is less than the second inner diameter. Further, in examples, the catheter further comprises an elongate movement member slidably disposed in a wall lumen in the elongate shaft. The elongate movement member can be coupled to the first portion. The instrument base can be configured to manipulate the elongate movement member to control actuation of the elongate shaft.

In some embodiments, the distal section of the elongate shaft is removably coupled to the middle section of the elongate shaft.

In some implementations, the present disclosure relates to an aspiration catheter comprising an elongate shaft configured to couple to an aspiration system and an instrument handle coupled to the elongate shaft and configured to manipulate the elongate shaft to control actuation of the elongate shaft. The elongate shaft includes a proximal portion, a medial portion, a tip portion, and a lumen extending from the proximal portion to the tip portion. The medial portion includes a first inner diameter that is larger than a second inner diameter of the tip portion. The tip portion is configured to removably receive debris within a patient.

In some embodiments, the tip portion includes at least one of a counterbore or a countersink. Further, in some embodiments, a length of the tip portion is less than the second inner diameter of the tip portion. Moreover, in some embodiments, a ratio of the second inner diameter of the tip portion to the first inner diameter of the medial portion is within a range of 0.5 to 0.9.

In some embodiments, the aspiration catheter further comprises a pull wire slidably disposed in a wire lumen in the elongate shaft. The pull wire is coupled to the tip portion. The instrument handle is configured to manipulate the pull wire to control actuation of the elongate shaft.

In some embodiments, the tip portion includes a first portion and a second portion that is distal to the first portion. The first portion can include the second inner diameter. The second portion can include a third inner diameter that is larger than the second inner diameter. In examples, a length of the first portion is less than the second inner diameter. Further, in examples, the aspiration catheter further comprises a pull wire slidably disposed in a wire lumen in the elongate shaft. The pull wire can be coupled to the first portion. The instrument handle can be configured to manipulate the pull wire to control actuation of the elongate shaft.

In some embodiments, the tip portion is removably coupled to the medial portion.

In some implementations, the present disclosure relates to a catheter comprising an elongate shaft and an instrument handle coupled to the elongate shaft and configured to control actuation of the elongate shaft. The elongate shaft includes a first section, a second section that is distal to the first section, and a lumen. The first section includes a first inner diameter and at least a portion of the second section includes a second inner diameter that is smaller than the first inner diameter. The elongate shaft is configured to provide aspiration to a target site via the lumen.

In some embodiments, a ratio of the second inner diameter to the first inner diameter is within a range of 0.5 to 0.9. Further, in some embodiments, a longitudinal length of the second section is less than the second inner diameter. Moreover, in some embodiments, the catheter further comprises an elongate movement member coupled to the second section. The instrument handle can be configured to manipulate the elongate movement member to control actuation of the elongate shaft.

In some embodiments, the second section of the elongate shaft includes a first portion and a second portion that is distal to the first portion. The first portion can include the second inner diameter and the second portion can include a third inner diameter that is larger than the second inner diameter. In examples, a length of the first portion is less than the second inner diameter. Further, in examples, the catheter further comprises an elongate movement member slidably disposed in a wall lumen in the elongate shaft. The elongate movement member can be coupled to the first portion. The instrument handle can be configured to manipulate the elongate movement member to control actuation of the elongate shaft.

In some embodiments, the second section of the elongate shaft includes one or more orientation markings.

In some implementations, the present disclosure relates to a system comprising an elongate shaft including a proximal portion, a medial portion, a tip portion, and a first lumen extending from the proximal portion to the tip portion. The medial portion includes a first inner diameter that is larger than a second inner diameter of the tip portion. The first lumen is configured to couple to an aspiration system to provide aspiration to a target site via the first lumen. The system further comprises an elongate movement member slidably disposed in a second lumen in the elongate shaft. The elongate movement member is coupled to the tip portion and configured to control actuation of the elongate shaft.

In some embodiments, a ratio of the second inner diameter to the first inner diameter is within a range of 0.5 to 0.9. Further, in some embodiments, a length of the tip portion is less than the second inner diameter.

In some embodiments, the tip portion includes a first section and a second section that is distal to the first section. The first section can include the second inner diameter and the second section can include a third inner diameter that is larger than the second inner diameter. In examples, a longitudinal length of the first section is less than the second inner diameter.

In some embodiments, the tip portion has a fracture toughness greater than or equal to 2 MPa·m^{1/2}. Further, in some embodiments, the tip portion includes at least one of stainless steel, titanium, tungsten, aluminum alloy, iron alloy, steel alloy, titanium alloy, or tungsten alloy.

For purposes of summarizing the disclosure, certain aspects, advantages and features are described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the disclosure. In addition, various features of different disclosed embodiments can be combined to form additional embodiments, which are part of this disclosure. Throughout the drawings, reference numbers may be reused to indicate correspondence between reference elements.

FIG. 1 illustrates an example robotic medical system arranged for a diagnostic and/or therapeutic ureteroscopy procedure in accordance with one or more embodiments.

FIG. 2 illustrates an example robotic medical system arranged for a diagnostic and/or therapeutic bronchoscopy procedure in accordance with one or more embodiments.

FIG. 3 illustrates an example table-based robotic system in accordance with one or more embodiments.

FIG. 4 illustrates example medical system components that may be implemented in any of the medical systems of FIGS. 1-3 in accordance with one or more embodiments.

FIG. 5 illustrates an example catheter disposed in the kidney of a patient in accordance with one or more embodiments.

FIG. 6 illustrates an example catheter including a shaft and a handle in accordance with one or more embodiments.

FIG. 7A illustrates a side view of the shaft of the catheter from FIG. 6 in accordance with one or more embodiments.

FIG. 7B illustrates a cross-sectional view of the shaft of the catheter from FIG. 6 in accordance with one or more embodiments.

FIG. 8 illustrates a perspective view of the shaft of the catheter from FIG. 6 in accordance with one or more embodiments.

FIGS. 9A and 9B illustrate perspectives view of the distal end portion of the shaft from FIG. 6 in an example where elongate movement members are attached to the distal end portion using a loop structure in accordance with one or more embodiments.

FIGS. 10A and 10B illustrate perspectives view of the distal end portion of the shaft from FIG. 6 in another example where elongate movement members are individually attached to the distal end portion in accordance with one or more embodiments.

FIG. 11 illustrates an exploded view of an example filter structure of the shaft from FIG. 6 in accordance with one or more embodiments.

FIG. 12A illustrates a front view of the example filter structure in accordance with one or more embodiments.

FIG. 12B illustrates a rear view the example filter structure in accordance with one or more embodiments.

FIGS. 13-1 illustrates a cross-sectional view of an example distal end portion of a shaft of a catheter in accordance with one or more embodiments.

FIGS. 13-2 illustrates a cross-sectional view of another example distal end portion of a shaft of a catheter in accordance with one or more embodiments.

FIG. 14A illustrates a perspective view of one or more markings that can be implemented in some examples on a tip structure of a shaft in accordance with one or more embodiments.

FIG. 14B illustrates a front view of the one or more markings from FIG. 14A in accordance with one or more embodiments.

### DETAILED DESCRIPTION

The headings provided herein are for convenience only and do not necessarily affect the scope or meaning of the disclosure. Although certain embodiments and examples are disclosed below, the subject matter extends beyond the specifically disclosed embodiments to other alternative embodiments and/or uses and to modifications and equivalents thereof. Thus, the scope of the claims that may arise here from is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that are similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

The present disclosure relates to aspiration/irrigation catheters/devices. With respect to percutaneous-access devices and other medical devices relevant to the present disclosure, the term “device” is used according to its broad and ordinary meaning and may refer to any type of tool, instrument, assembly, system, apparatus, component, or the like. In some contexts herein, the term “instrument” may be used substantially interchangeably with the term “device.”

Although certain aspects of the present disclosure are described in detail herein in the context of renal, urological, and/or nephrological procedures, such as kidney stone removal/treatment procedures, it should be understood that such context is provided for convenience, and the concepts disclosed herein are applicable to any suitable medical procedures, such as a bronchoscopy. However, as mentioned, description of the renal/urinary anatomy and associated medical issues and procedures is presented below to aid in the description of the concepts disclosed herein.

Kidney stone disease, also known as urolithiasis, is a medical condition that involves the formation in the urinary tract of a solid piece of material, referred to as “kidney stones,” “urinary stones,” “renal calculi,” “renal lithiasis,” or “nephrolithiasis.” Urinary stones may be formed and/or found in the kidneys, the ureters, and the bladder (referred to as “bladder stones”). Such urinary stones can form as a result of mineral concentration in urinary fluid and can cause significant abdominal pain once such stones reach a size sufficient to impede urine flow through the ureter or urethra. Urinary stones may be formed from calcium, magnesium, ammonia, uric acid, cystine, and/or other compounds or combinations thereof.

Several methods can be used for treating patients with kidney stones, including observation, medical treatments (such as expulsion therapy), non-invasive treatments (such as extracorporeal shock wave lithotripsy (ESWL)), minimally-invasive or surgical treatments (such as ureteroscopy and percutaneous nephrolithotomy (“PCNL”)), and so on. In some approaches (e.g., ureteroscopy and PCNL), the physician gains access to the stone, the stone is broken into smaller pieces or fragments, and the relatively small stone fragments/particulates are extracted from the kidney using a basketing device and/or aspiration.

In ureteroscopy procedures, a physician may insert a ureteroscope into the urinary tract through the urethra to remove urinary stones from the bladder and ureter. Typically, a ureteroscope includes an imaging device at its distal end configured to enable visualization of the urinary tract. The ureteroscope can also include a lithotripsy device to capture or break apart urinary stones. During a ureteroscopy procedure, one physician/technician may control the position of the ureteroscope, while another other physician/technician may control the lithotripsy device(s).

In PCNL procedures, which may be used to remove relatively large stones, a physician may insert a nephroscope through the skin (i.e., percutaneously) and intervening tissue to provide access to the treatment site for breaking-up and/or removing the stone(s). During PCNL procedures, fluidics can be applied to clear stone dust, small fragments, and/or thrombus from the treatment site and/or the visual field. In some instances, a relatively straight and/or rigid nephroscope is used, wherein the physician positions the tip of the nephroscope at the appropriate location within the kidney (e.g., calyx) by pushing/leveraging the device against the patient's body. This movement can be harmful to the patient (e.g., cause tissue damage).

In other procedures, such as one or more of those discussed in further detail below, a physician can use multiple instruments via a percutaneous and/or direct access path to remove a kidney stone. For example, a physician can navigate a scope to a target site in a kidney through the urethra in a patient and insert a catheter device into the target site through the skin of the patient. The physician can use the scope and the catheter device in cooperation to fragment the kidney stone and extract the fragments from the patient.

In some instances of using a device to remove a kidney stone (in the above noted procedures or other procedures), a stone fragment may be relatively large and become stuck in the device. For example, upon fragmenting a stone into pieces, a stone piece may still be too large to make it through an entirety of a shaft/tube of a removal device. Further, a stone piece (having an oblong or other shape) may initially be positioned with the appropriate orientation to travel into a shaft/tube, but become clogged in the shaft/tube when the orientation changes. In any event, a stone/stone fragment that becomes clogged in a medical device can adversely affect the integrity of the medical device and/or the efficacy of the procedure, such as by preventing other stone pieces from being removed, damaging the medical device, and so on.

The present disclosure relates to systems, devices, and methods for navigating to and/or aspirating/irrigating a target site to perform a medical procedure. For example, a catheter can be implemented that includes an elongate shaft and a handle/base coupled to the shaft and configured to control actuation of the shaft (at least at a distal portion of the shaft). The shaft can include a lumen configured to couple to an aspiration/irrigation system to provide aspiration/irrigation to a target site, such as to remove an object from a patient. The handle/base of the catheter can be controlled robotically and/or manually to articulate the distal portion of the shaft, so that the catheter can be navigated within the anatomy of a patient. For instance, the catheter can include multiple pull wires or other elongate movement members that are coupled to the distal portion of the shaft and one or more manipulation components in the handle of the catheter. The pull wires/elongate movement members can be manipulated (using the handle) to control movement of the distal portion of the shaft. Additionally, or alternatively, the handle of the catheter can be moved to control movement of the distal portion of the catheter, such as to insert/retract/roll the tip of the catheter.

In some examples, the distal portion of the catheter includes a filter section to prevent objects greater than a particular size from entering the shaft of the catheter. For instance, the shaft of the catheter can include a proximal portion, a medial portion, a distal tip portion, and a lumen extending from the proximal portion to the tip portion. The medial portion can include a first inner diameter that is larger than a second inner diameter of the distal tip portion to prevent objects from entering the medial portion of the shaft. In some implementations, the distal tip portion has a length that is less than the second inner diameter of the distal tip portion to further prevent objects greater than a particular size from entering into the shaft of the catheter. This may also prevent certain oblong-shaped objects from passing into the shaft. In some implementations, the distal tip portion includes a control section having the smaller inner diameter (i.e., the second inner diameter to filter objects) and a counterbore/countersink section located distally to the control section, which has a larger inner diameter. This may allow an object to be held by the catheter without passing into the shaft, such as to maintain a kidney stone in a relatively fixed position while another instrument (e.g., laser, ultrasonic fragmenting device, lithotripter device, etc.) fragments the stone into pieces.

In some embodiments, the techniques and devices discussed herein can enable objects to be removed from patients in an efficient manner that prevents damage to the anatomy of the patients and/or damage to the removal devices. For example, the articulable catheter structures discussed herein can enable a physician to navigate a distal portion of a catheter within a patient without moving an entirety of the catheter (e.g., by controlling one or more elements within a handle/base of the catheter). In contrast, some nephoscopy procedures require a physician to leverage a proximal portion of a nephroscope to place a tip of the nephroscope in the appropriate location within the patient, resulting in damage to the anatomy of the patient. Further, the techniques and devices discussed herein can provide filtering functionality to avoid having undesired objects enter into the devices. For example, a distal portion of a catheter can include a filter section having a particular inner diameter and/or length to prevent objects of undesired sizes from entering to the rest of the shaft of the catheter. Moreover, the distal portion of the catheter can include a counterbore/countersink section to hold an object, such as while another device treats the object.

In some implementations, the techniques discussed herein implement robotic-assisted medical procedures, wherein robotic tools enable a physician to perform endoscopic and/or percutaneous access and/or treatment for a target anatomical site. For example, the robotic tools can engage with and/or control one or more medical instruments, such as a scope, catheter, or another instrument, to access a target site in a patient and/or perform a treatment at the target site. In some cases, the robotic tools are guided/controlled by a physician. In other cases, the robotic tools operate in an automatic or semi-automatic manner. Although some techniques are discussed in the context of robotic-assisted medical procedures, the techniques may be applicable to other types of medical procedures, such as procedures that do not implement robotic tools or implement robotic tools for relatively few operations (e.g., less than a threshold number). For example, the techniques can be applicable to procedures in which a manually operated medical instrument is implemented, such as a manual catheter and/or scope controlled entirely by a physician.

Certain aspects of the present disclosure are described herein in the context of renal, urological, and/or nephrological procedures, such as kidney stone removal/treatment procedures. However, it should be understood that such context is provided for convenience, and the concepts disclosed herein are applicable to any suitable medical procedure. For example, the following description is also applicable to other surgical/medical operations or medical procedures concerned with the removal of objects from a patient, including any object that can be removed from a treatment site or patient cavity (e.g., the esophagus, ureter, intestine, eye, etc.) via percutaneous and/or endoscopic access, such as, for example, gallbladder stone removal, lung (pulmonary/transthoracic) tumor biopsy, cataract removal, etc. However, as mentioned, description of the renal/urinary anatomy and associated medical issues and procedures is presented below to aid in the description of the concepts disclosed herein.

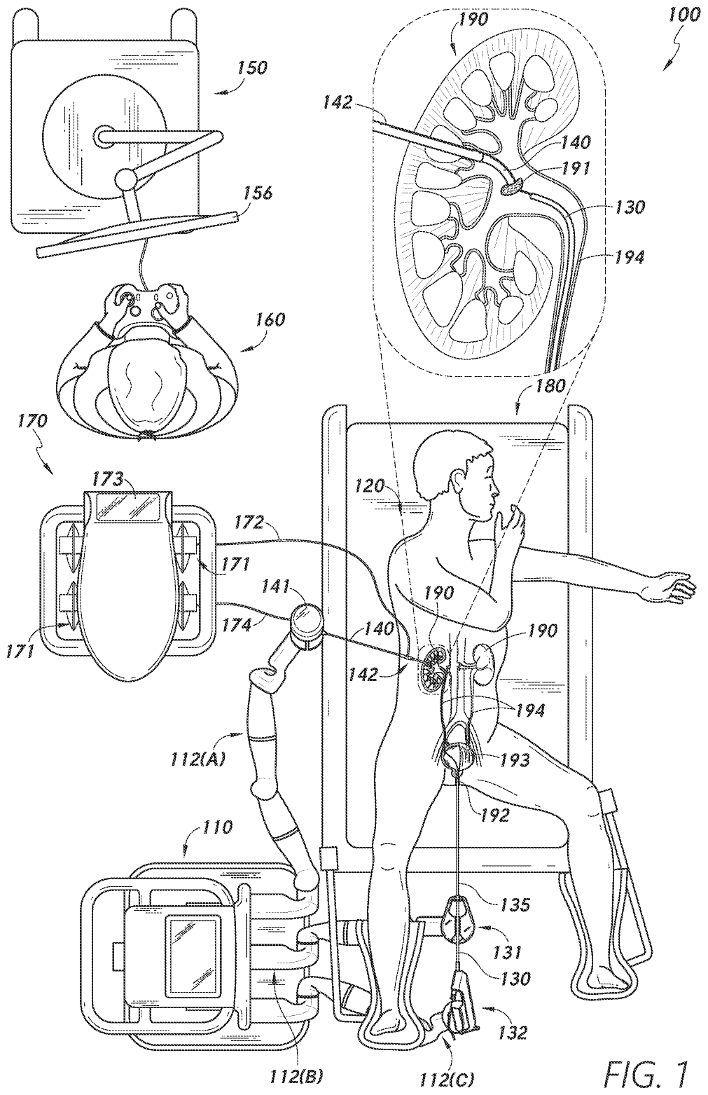

FIG. 1 illustrates an example robotic medical system 100 arranged for a diagnostic and/or therapeutic ureteroscopy procedure in accordance with one or more embodiments. The medical system 100 includes a robotic system 110 configured to engage with and/or control one or more medical instruments/devices to perform a procedure on a patient 120. In the example of FIG. 1, the robotic system 110 couples to a scope 130 and a catheter 140. However, the robotic system 110 can couple to any type of medical instrument. The medical system 100 also includes a control system 150 configured to interface with the robotic system 110 and/or a physician 160, provide information regarding the procedure, and/or perform a variety of other operations. For example, the control system 150 can include a display(s) 156 configured to present certain information to assist the physician 160 in performing the procedure. The medical system 100 can also include a fluid management system 170 (sometimes referred to as “the aspiration system 170” or “the irrigation system 170”) configured to provide aspiration and/or irrigation to a target site, such as via the catheter 140, the scope 130, an instrument/device 142, and/or another instrument/device. The medical system 100 can include a table 180 (e.g., bed) to hold the patient 120. Various acts are described herein as being performed by the physician 160. These acts can be performed directly by the physician 160, a user under the direction of the physician 160, another user (e.g., a technician), a combination thereof, and/or any other user. The devices/components of the medical system 100 can be arranged in a variety of ways depending on the type procedure, phase of the procedure, user preferences, and so on.

The control system 150 can generally operate in cooperation with the robotic system 110 to perform the medical procedure. For example, the control system 150 can communicate with the robotic system 110 via a wireless or wired connection to control a medical instrument connected to the robotic system 110, receive an image(s) captured by a medical instrument, and so on. For example, the control system 150 can receive image data from the scope 130 (e.g., an imaging device associated with the scope 130) and display the image data (and/or representations generated therefrom) to the physician 160 to assist the physician 160 in navigating the scope 130 and/or the catheter 140 within the patient 120. The physician 160 can provide input via an input/output (I/O) device, such as a controller, and the control system 150 can send control signals to the robotic system 110 to control movement of the scope 130/catheter 140 connected to the robotic system 110. The scope 130/catheter 140 (and/or another medical instrument) can be configured to move in a variety of manners, such as to articulate, roll, and so on.

In some embodiments, the control system 150 can provide power to the robotic system 110 via one or more electrical connections, provide optics to the robotic system 110 via one or more optical fibers or other components, and so on. In examples, the control system 150 can communicate with a medical instrument to receive sensor data (via the robotic system 110 and/or directly from the medical instrument). Sensor data can indicate or be used to determine a position and/or orientation of the medical instrument. Further, in examples, the control system 150 can communicate with the table 180 to position the table 180 in a particular orientation or otherwise control the table 180. Moreover, in examples, the control system 150 can communicate with an EM field generator (not illustrated) to control generation of an EM field around the patient 120.

The robotic system 110 can include one or more robotic arms 112 configured to engage with and/or control a medical instrument(s)/device. Each robotic arm 112 can include multiple arm segments coupled to joints, which can provide multiple degrees of movement. A distal end of a robotic arm 112 (e.g., end effector) can be configured to couple to an instrument/device. In the example of FIG. 1, the robotic arm 112(A) is coupled to a handle 141 of the catheter 140. The second robotic arm 112(B) is coupled to a scope-driver instrument coupling/device 131, which can facilitate robotic control/advancement of the scope 130. Further, the third robotic arm 112(C) is coupled to a handle 132 of the scope 130, which can be configured to facilitate advancement and/or operation of the scope 130 and/or a medical instrument that can be deployed through the scope 130, such as an instrument deployed through a working channel of the scope 130. In this example, the second robotic arm 112(B) and/or the third robotic arm 112(C) can control movement of the scope 130 (e.g., articulation, roll, etc.). Although three robotic arms are connected to particular medical instruments in FIG. 1, the robotic system 110 can include any number of robotic arms that are configured to connect to any type of medical instrument/device.

The robotic system 110 can be communicatively coupled to any component of the medical system 100. For example, the robotic system 110 can be communicatively coupled to the control system 150 to receive a control signal from the control system 150 to perform an operation, such as to control a robotic arm 112 in a particular manner, manipulate a medical instrument, and so on. Further, the robotic system 110 can be configured to receive an image (also referred to as image data) from the scope 130 depicting internal anatomy of the patient 120 and/or send the image to the control system 150, which can then be displayed on the display(s) 156. Moreover, the robotic system 110 can be coupled to a component of the medical system 100, such as the control system 150 and/or the fluid management system 170, in a manner as to allow for fluids, optics, power, data, or the like to be received therefrom.

The fluid management system 170 can be configured to provide/control aspiration and/or irrigation to a target site. As shown, the fluid management system 170 can be configured to hold one or more fluid bags/containers 171 and/or control fluid flow thereto/therefrom. For example, an irrigation line 172 may be coupled to one or more of the bags/containers 171 and to an irrigation port of a percutaneous-access device/assembly 142. Irrigation fluid may be provided to the target anatomy via the irrigation line 172 and the percutaneous-access device/assembly 142. The fluid management system 170 may include certain electronic components, such as a display 173, flow control mechanics, and/or certain associated control circuitry. The fluid management cart 170 may comprise a stand-alone tower/cart and may have one or more IV bags 171 hanging on one or more sides thereof. The cart 170 may include a pump with which aspiration fluid may be pulled into a collection container/cartridge via an aspiration channel/tube 174. The aspiration channel/tube 174 may be coupled to the catheter handle 141 to facilitate aspiration via a lumen in the catheter 140.

In the illustrated system 100, the percutaneous-access device 142 is implemented to provide percutaneous access to a kidney 190 of the patient 120. The percutaneous-access instrument 142 may include one or more sheaths and/or shafts through which instruments and/or fluids may access the target anatomy in which the distal end of the instrument 142 is disposed. In this example, the catheter 140 accesses the renal anatomy through the percutaneous-access device 142. That is, the catheter 140 is inserted into the instrument 142 to access the target site.

Although various examples are discussed in the context of providing irrigation/aspiration via the catheter 140 and/or the percutaneous-access device/assembly 142, irrigation fluid and/or aspiration may be provided to the treatment site (e.g., kidney) through another device, such as the scope 130, in some cases. Furthermore, irrigation and aspiration may or may not be provided through the same instrument(s). Where one or more of instruments provides the irrigation and/or aspiration functionality, one or more others of the instruments may be used for other functionality, such as breaking-up the object to be removed.

A medical instrument can include a variety of types of instruments, such as a scope (sometimes referred to as an “endoscope”), a catheter, a needle, a guidewire, a lithotripter, a basket retrieval device, forceps, a vacuum, a needle, a scalpel, an imaging probe, an imaging device, jaws, scissors, graspers, needle holder, micro dissector, staple applier, tacker, suction/irrigation tool, clip applier, and so on. A medical instrument can include a direct entry instrument, percutaneous entry instrument, and/or another type of instrument. In some embodiments, a medical instrument is a steerable device, while in other embodiments a medical instrument is a non-steerable device. In some embodiments, a surgical tool refers to a device that is configured to puncture or to be inserted through the human anatomy, such as a needle, a scalpel, a guidewire, and so on. However, a surgical tool can refer to other types of medical instruments.

The term “scope” or “endoscope” can refer to any type of elongate medical instrument having image generating, viewing, and/or capturing functionality (or configured to provide such functionality with an imaging device deployed though a working channel) and configured to be introduced into any type of organ, cavity, lumen, chamber, and/or space of a body. For example, a scope or endoscope, such as the scope 130, can refer to a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), a borescope, and so on. A scope/endoscope, in some instances, may comprise a rigid or flexible tube and/or may be dimensioned to be passed within an outer sheath, catheter, introducer, or other lumen-type device, or may be used without such devices. In some embodiments, a scope includes one or more working channels through which additional tools/medical instruments, such as lithotripters, basketing devices, forceps, laser devices, imaging devices, etc., can be introduced into a treatment site.

The terms “direct entry” or “direct access” can refer to any entry of instrumentation through a natural or artificial opening in a patient's body. For example, the scope 130 may be referred to as a direct access instrument, since the scope 130 enters into the urinary tract of a patient via the urethra.

The terms “percutaneous entry” or “percutaneous access” can refer to entry, such as by puncture and/or minor incision, of instrumentation through the skin of a patient and any other body layers necessary to reach a target anatomical location associated with a procedure (e.g., the calyx network of the kidney). As such, a percutaneous access instrument may refer to a medical instrument, device, or assembly that is configured to puncture or to be inserted through skin and/or other tissue/anatomy, such as a needle, scalpel, guidewire, sheath, shaft, scope, catheter, and the like. However, it should be understood that a percutaneous access instrument can refer to other types of medical instruments in the context of the present disclosure. In some embodiments, a percutaneous access instrument refers to an instrument/device that is inserted or implemented with a device that facilitates a puncture and/or minor incision through the skin of a patient. For example, the catheter 140 may be referred to as a percutaneous access instrument when the catheter 140 is inserted through a sheath/shaft that is inserted into the skin of a patient.

In some embodiments, a medical instrument includes a sensor (also referred to as a “position sensor”) that is configured to generate sensor data. In examples, sensor data can indicate a position and/or orientation of the medical instrument and/or can be used to determine a position and/or orientation of the medical instrument. For instance, sensor data can indicate a position and/or orientation of a scope, which can indicate a roll of a distal end of the scope. A position and orientation of a medical instrument can be referred to as a pose of the medical instrument. A sensor can be positioned on a distal end of a medical instrument and/or any other location. In some embodiments, a sensor can provide sensor data to the control system 150, the robotic system 110, and/or another system/device to perform one or more localization techniques to determine/track a position and/or an orientation of a medical instrument.

In some embodiments, a sensor can include an electromagnetic (EM) sensor with a coil of conductive material. Here, an EM field generator can provide an EM field that is detected by the EM sensor on the medical instrument. The magnetic field can induce small currents in coils of the EM sensor, which can be analyzed to determine a distance and/or angle/orientation between the EM sensor and the EM field generator. Further, a sensor can include another type of sensor, such as a camera, a range sensor (e.g., depth sensor), a radar device, a shape sensing fiber, an accelerometer, a gyroscope, an accelerometer, a satellite-based positioning sensor (e.g., a global positioning system (GPS)), a radio-frequency transceiver, and so on.

In some embodiments, the medical system 100 can also include an imaging device (not illustrated in FIG. 1) which can be integrated into a C-arm and/or configured to provide imaging during a procedure, such as for a fluoroscopy-type procedure. The imaging device can be configured to capture/generate one or more images of the patient 120 during a procedure, such as one or more x-ray or CT images. In examples, images from the imaging device can be provided in real-time to view anatomy and/or medical instruments within the patient 120 to assist the physician 160 in performing a procedure. The imaging device can be used to perform a fluoroscopy (e.g., with a contrast dye within the patient 120) or another type of imaging technique.

The various components of the medical system 100 can be communicatively coupled to each other over a network, which can include a wireless and/or wired network. Example networks include one or more personal area networks (PANs), local area networks (LANs), wide area networks (WANs), Internet area networks (LANs), body area networks (BANs), cellular networks, the Internet, etc. Further, in some embodiments, the components of the medical system 100 are connected for data communication, fluid/gas exchange, power exchange, and so on, via one or more support cables, tubes, or the like.

In some examples, the medical system 100 is implemented to perform a medical procedure relating to the renal anatomy, such as to treat kidney stones. For instance, robotic-assisted percutaneous procedures can be implemented, wherein robotic tools (e.g., one or more components of the medical system 100) can enable a physician/urologist to perform endoscopic (e.g., ureteroscopy) target access as well as percutaneous access/treatment. This disclosure, however, is not limited to kidney stone removal and/or robotic-assisted procedures. In some implementations, robotic medical solutions can provide relatively higher precision, superior control, and/or superior hand-eye coordination with respect to certain instruments compared to strictly manual procedures. For example, robotic-assisted percutaneous access to the kidney in accordance with some procedures can advantageously enable a urologist to perform both direct-entry endoscopic renal access and percutaneous renal access. Although some embodiments of the present disclosure are presented in the context of catheters, nephroscopes, ureteroscopes, and/or the human renal anatomy, it should be understood that the principles disclosed herein may be implemented in any type of endoscopic/percutaneous procedure or another type of procedure.

In one illustrative and non-limiting procedure, the medical system 100 can be used to remove a kidney stone 191 from the patient 120. During setup for the procedure, the physician 160 can position the robotic arms 112 of the robotic system 110 in the desired configuration and/or attach the appropriate medical instruments. For example, the physician 160 can position the first robotic arm 112(A) near a treatment site and attach an EM field generator (not illustrated), which can assist in tracking a location of the scope 130 and/or other instruments/devices during the procedure. Further, the physician 160 can position the second robotic arm 112(B) between the legs of the patient 120 and attach the scope-driver instrument coupling 131, which can facilitate robotic control/advancement of the scope 130. In some instances, the physician 160 can insert a sheath/access instrument 135 into the urethra 192 of the patient 120 and/or through the bladder 193 and up the ureter 194. The physician 160 can connect the sheath/access instrument 135 to the scope-drive instrument coupling 131. The sheath/access instrument 135 can include a lumen-type device configured to receive the scope 130, thereby assisting in inserting the scope 130 into the anatomy of the patient 120. However, in some embodiments the sheath/access instrument 135 is not used (e.g., the scope 130 is inserted directly into the urethra 192). The physician 160 can then insert the scope 130 into the sheath/access 135 instrument manually, robotically, or a combination thereof. The physician 160 can attach the handle 132 of the scope 130 to the third robotic arm 112(C), which can be configured to facilitate advancement and/or operation of a basketing device, laser device, and/or another medical instrument deployed through the scope 130.

The physician 160 can interact with the control system 150 to cause the robotic system 110 to advance and/or navigate the scope 130 into the kidney 190. For example, the physician 160 can navigate the scope 130 using a controller or other I/O device to locate the kidney stone 191. The control system 150 can provide information via the display(s) 156 regarding the scope 130 to assist the physician 160 in navigating the scope 130, such as to view an image representation (e.g., a real-time image(s) captured by the scope 130). In some embodiments, the control system 150 can use localization techniques to determine a position and/or an orientation of the scope 130, which can be viewed by the physician 160 through the display(s) 156, in some cases. Further, other types of information can also be presented through the display(s) 156 to assist the physician 160 in controlling the scope 130, such as x-ray images of the internal anatomy of the patient 120.

Once at the site of the kidney stone 191 (e.g., within the calyx of the kidney 190), the scope 130 can be used to designate/tag a target location for a catheter to access the kidney 190 percutaneously. To minimize damage to the kidney 190 and/or the surrounding anatomy, the physician 160 can designate a papilla as the target location for entering into the kidney 190 percutaneously. However, other target locations can be designated or determined. In some embodiments of designating the papilla, the physician 160 can navigate the scope 130 to contact the papilla, the control system 150 can use localization techniques to determine a location of the scope 130 (e.g., a location of the distal end of the scope 130), and the control system 150 can associate the location of the scope 130 with the target location. Further, in some embodiments, the physician 160 can navigate the scope 130 to be within a particular distance to the papilla (e.g., park in front of the papilla) and provide input indicating that the target location is within a field-of-view of the scope 130. The control system 150 can perform image analysis and/or other localization techniques to determine a location of the target location. Moreover, in some embodiments, the scope 130 can deliver a fiduciary to mark the papilla as the target location.

When the target location is designated, the catheter 140 can be inserted through a percutaneous access path into the patient 120 to reach the target site (e.g., rendezvous with the scope 130). For example, the catheter 140 can be connected to the first robotic arm 112(A) (upon removing the EM field generator) and the physician 160 can interact with the control system 150 to cause the robotic system 110 to advance and/or navigate the catheter 140, as shown in FIG. 1. Alternatively, or additionally, the catheter 140 can be manually inserted and/or controlled, such as when the catheter 140 is implemented as a manually-controllable catheter. In some embodiments, a needle or another medical instrument is inserted into the patient 120 to create the percutaneous access path. The control system 150 can provide information via the display(s) 156 regarding the catheter 140 to assist the physician 160 in navigating the catheter. For example, the display(s) 156 can provide image data from the perspective of the scope 130, wherein the image data may depict the catheter 140 (e.g., when within the field-of-view of an imaging device of the scope 130).

With the scope 130 and/or the catheter 140 located at the target location, the physician 160 can use the scope 130 to break up the kidney stone 191 and/or use the catheter 140 to extract pieces of the kidney stone 191 from the patient 120. For example, the scope 130 can deploy a tool (e.g., a laser, a cutting instrument, lithotripter, etc.) through a working channel to fragment the kidney stone 191 into pieces and the catheter 140 can suck out the pieces from the kidney 190 through the percutaneous access path. The catheter 140 can provide aspiration to maintain/hold the kidney stone 191 at a distal end of the catheter 140 and/or at a relatively fixed position, while the scope 130 fragments the kidney stone 191 using a tool (e.g., laser), as shown in FIG. 1. The fluid management system 170 can provide irrigation to the target site via the percutaneous-access device/assembly 142 and/or provide aspiration to the target site via the catheter 140 (e.g., a lumen in the catheter 140).

Although various example procedures are discussed in the context of implementing a robotically controlled catheter 140, the procedure can be implemented with a manually controllable catheter. For example, the catheter 140 can include a manually controllable handle that is configured to be held/manipulated by the physician 160. The physician 160 can navigate the catheter 140 by rolling, inserting, retracting, or otherwise manipulating the handle and/or a manual actuator, which can result in articulation of a distal portion of the catheter 140. Example robotically controllable and manually controllable catheters are discussed in further detail below.

The medical system 100 (and/or other medical systems discussed herein) can provide a variety of benefits, such as providing guidance to assist a physician in performing a procedure (e.g., instrument tracking, instrument navigation, instrument calibration, etc.), enabling a physician to perform a procedure from an ergonomic position without the need for awkward arm motions and/or positions, enabling a single physician to perform a procedure with one or more medical instruments, avoiding radiation exposure (e.g., associated with fluoroscopy techniques), enabling a procedure to be performed in a single-operative setting, providing continuous aspiration/irrigation to remove an object more efficiently (e.g., to remove a kidney stone), and so on. For example, the medical system 100 can provide guidance information to assist a physician in using various medical instruments to access a target anatomical feature while minimizing bleeding and/or damage to anatomy (e.g., critical organs, blood vessels, etc.). Further, the medical system 100 can provide non-radiation-based navigational and/or localization techniques to reduce physician and patient exposure to radiation and/or reduce the amount of equipment in the operating room. Moreover, the medical system 100 can provide functionality that is distributed between at least the control system 150 and the robotic system 110, which can be independently movable. Such distribution of functionality and/or mobility can enable the control system 150 and/or the robotic system 110 to be placed at locations that are optimal for a particular medical procedure, which can maximize working area around the patient and/or provide an optimized location for a physician to perform a procedure.

Although various techniques/systems are discussed as being implemented as robotically-assisted procedures (e.g., procedures that at least partly use the medical system 100), the techniques/systems can be implemented in other procedures, such as in fully-robotic medical procedures, human-only procedures (e.g., free of robotic systems), and so on. For example, the medical system 100 can be used to perform a procedure without a physician holding/manipulating a medical instrument and without a physician controlling movement of a robotic system/arm (e.g., a fully-robotic procedure that relies on relatively little input to direct the procedure). That is, medical instruments that are used during a procedure can each be held/controlled by components of the medical system 100, such as the robotic arms 112 of the robotic system 110.

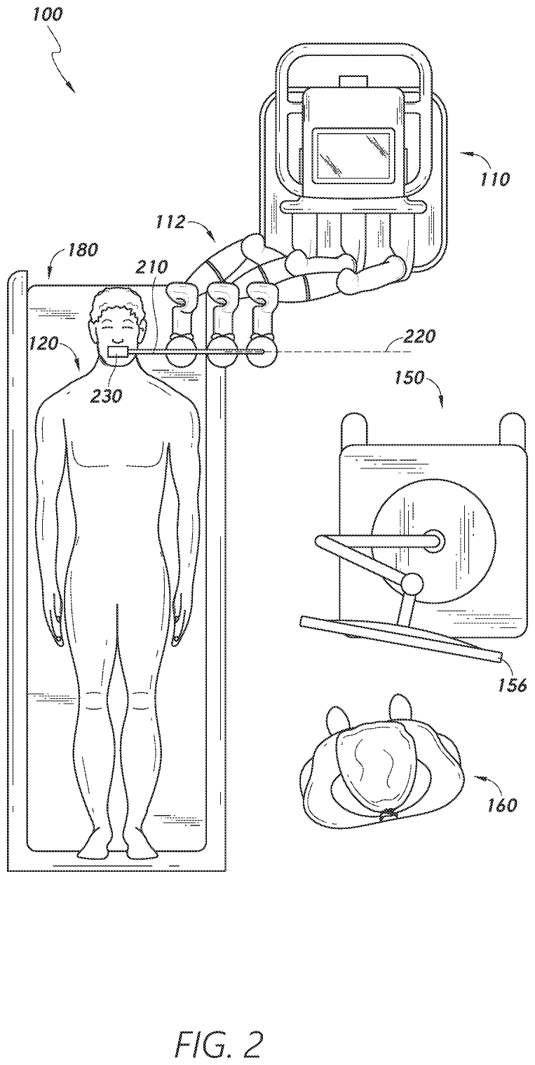

FIG. 2 illustrates the example robotic medical system 100 arranged for a diagnostic and/or therapeutic bronchoscopy procedure in accordance with one or more embodiments. During a bronchoscopy, the arm(s) 112 of the robotic system 110 may be configured to deliver a medical instrument, such as a steerable endoscope 210, which may be a procedure-specific bronchoscope for bronchoscopy, to a natural orifice access point (i.e., the mouth of the patient 120 positioned on the table 180 in the present example) to deliver diagnostic and/or therapeutic tools. As shown, the robotic system 110 (e.g., cart) may be positioned proximate to the patient's upper torso in order to provide access to the access point. Similarly, the robotic arms 112 may be actuated to position the bronchoscope 210 relative to the access point. The arrangement in FIG. 2 may also be utilized when performing a gastro-intestinal (GI) procedure with a gastroscope, a specialized endoscope for GI procedures.

Once the robotic system 110 is properly positioned, the robotic arms 112 may insert the steerable endoscope 210 into the patient robotically, manually, or a combination thereof. The steerable endoscope 210 may comprise at least two telescoping parts, such as an inner leader portion and an outer sheath portion, with each portion coupled to a separate instrument driver from a set of instrument drivers and/or with each instrument driver coupled to the distal end of a respective robotic arm 112. This linear arrangement of the instrument drivers creates a “virtual rail” 220 that may be repositioned in space by manipulating the one or more robotic arms 112 into different angles and/or positions. The virtual rails/paths described herein are depicted in the figures using dashed lines that generally do not depict any physical structure of the system. Translation of one or more of the instrument drivers along the virtual rail 220 can advance or retract the endoscope 210 from the patient 120.

The endoscope 210 may be directed down the patient's trachea and lungs after insertion using precise commands from the robotic system 110 until reaching the target operative site. The use of separate instrument drivers can allow independent driving of separate portions of the endoscope/assembly 210. For example, the endoscope 210 may be directed to deliver a biopsy needle to a target, such as, for example, a lesion or nodule within the lungs of a patient. The needle may be deployed down a working channel that runs the length of the endoscope 210 to obtain a tissue sample to be analyzed by a pathologist. Depending on the pathology results, additional tools may be deployed down the working channel of the endoscope 210 for additional biopsies. For example, when a nodule is identified as being malignant, the endoscope 210 may endoscopically deliver tools to resect the potentially cancerous tissue. In some instances, diagnostic and therapeutic treatments can be delivered in separate procedures. In those circumstances, the endoscope 210 may also be used to deliver a fiducial to “mark” the location of the target nodule as well. In other instances, diagnostic and therapeutic treatments may be delivered during the same procedure.

In the arrangement of the system 100 in FIG. 2, a patient introducer 230 is attached to the patient 120 via a port (not shown; e.g., surgical tube). The patient introducer 230 may be secured to the table 180 (e.g., via a patient introducer holder configured to support the introducer 230 and secure the position of the patient introducer 230 with respect to the table 180 or other structure). In some embodiments, the patient introducer 230 may include a proximal end, a distal end, and an introducer tube therebetween. The proximal end of the patient introducer 230 can provide an opening/orifice which may be configured to receive the instrument 210 (e.g., bronchoscope), and the distal end of the patient introducer 230 can provide a second opening which may be configured to guide the instrument 210 into the patient-access port. A curved tube component of the introducer 230 can connect the proximal and distal ends thereof and guide the instrument 210 through the introducer 230.

The curvature of the introducer 230 may enable the robotic system 110 to manipulate the instrument 210 from a position that is not in direct axial alignment with the patient-access port, thereby allowing for greater flexibility in the placement of the robotic system 110 within the room. Further, the curvature of the introducer 230 may allow the robotic arms 112 of the robotic system 110 to be substantially horizontally aligned with the patient introducer 230, which may facilitate manual movement of the robotic arm(s) 112 if needed.

In some embodiments, one or more of the catheters discussed herein can be implemented in a bronchoscopy procedure, such as that illustrated in FIG. 2. For example, a catheter can be implemented in cooperation with or instead of the endoscope 210 to remove an object from the patient 120. In one illustration, a catheter and the endoscope 210 are interchanged on the robotic arms 112 and separately used to investigate/treat a target site. Here, the catheter can be inserted through the patient introducer 230 and used to provide aspiration/irrigation, such as to remove an object from the patient 120. In another illustration, a catheter is deployed through a working channel on the endoscope 210 to provide irrigation/aspiration.

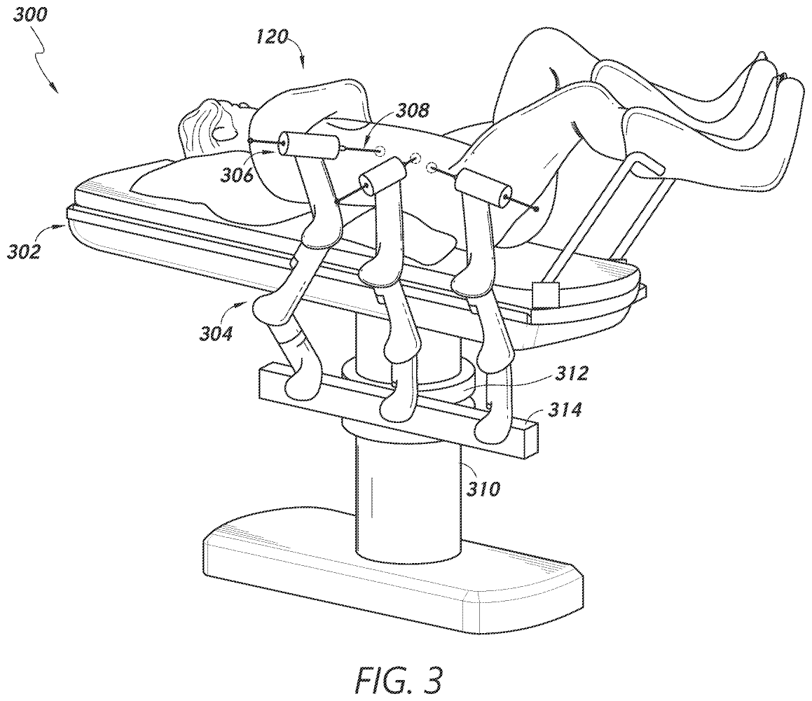

FIG. 3 illustrates a table-based robotic system 300 configured to perform a medical procedure in accordance with one or more embodiments. Here, one or more of the robotic components of the robotic medical system 100 can be incorporated into a table 302, which can reduce the amount of capital equipment within an operating room and/or allow greater access to the patient 120, in comparison to cart-based robotic systems. For example, the system 300 can include one or more components of the control system 150, the robotic system 110, and/or the fluid management system 170.

As shown, the table 302 can include/incorporate one or more robotic arms 304 configured to engage with and/or control a medical instrument(s)/device. Each robotic arm 304 can include multiple arm segments coupled to joints, which can provide multiple degrees of movement. A distal end of a robotic arm 304 (i.e., end effector 306) can be configured to couple to an instrument/device, which can include any of the medical instruments/devices discussed herein, such as a catheter, needle, scope, etc. Each robotic arm 304 can be similar to or different than the robotic arms 112 of the system 100 of FIGS. 1 and 2. Further, each end effector 306 can be similar to or different than an end effector of the robotic system 100.

As shown, the robotic-enabled table system 300 can include a column 310 coupled to one or more carriages 312 (e.g., ring-shaped movable structures), from which the one or more robotic arms 304 may emanate. The carriage(s) 312 may translate along a vertical column interface that runs at least a portion of the length of the column 310 to provide different vantage points from which the robotic arms 304 may be positioned to reach the patient 120. The carriage(s) 312 may rotate around the column 310 in some embodiments using a mechanical motor positioned within the column 310 to allow the robotic arms 304 to have access to multiples sides of the table 302. Rotation and/or translation of the carriage(s) 312 can allow the system 300 to align the medical instruments, such as endoscopes and/or catheters, into different access points on the patient 120. By providing vertical adjustment, the robotic arms 304 can be configured to be stowed compactly beneath the platform of the table system 300 and subsequently raised during a procedure. The robotic arms 304 may be mounted on the carriage(s) 312 through one or more arm mounts 314, which may comprise a series of joints that may individually rotate and/or telescopically extend to provide additional configurability to the robotic arms 304. The column 310 structurally provides support for the table platform and a path for vertical translation of the carriage(s) 312. The column 310 may also convey power and control signals to the carriage(s) 312 and/or the robotic arms 304 mounted thereon.

In some embodiments, the table-based robotic system 300 can include or be associated with a control system, similar to the control system 150, to interface with a physician and/or provide information regarding a medical procedure. For example, a control system can include an input component(s) to enable a physician to control the one or more robotic arms 304 and/or medical instruments attached to the one or more robotic arms 304. In some implementations, the input component(s) enables the physician to provide input to control a medical instrument in a similar manner as if the physician were physically holding/manipulating the medical instrument.

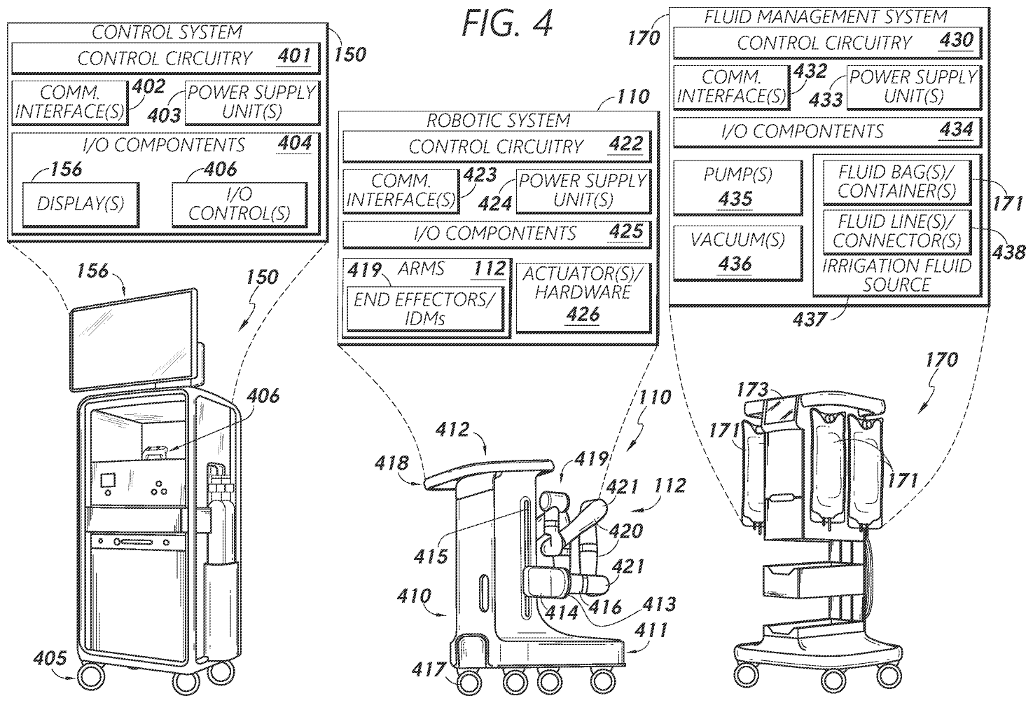

FIG. 4 illustrates medical system components that may be implemented in any of the medical systems of FIGS. 1-3 in accordance with one or more embodiments of the present disclosure. Although certain components in FIG. 4, it should be understood that additional components not shown can be included in embodiments in accordance with the present disclosure. Furthermore, any of the illustrated components can be omitted, interchanged, and/or integrated into other devices/systems, such as the table 180, a medical instrument, etc.

The control system 150 can include one or more of the following components, devices, modules, and/or units (referred to herein as “components”), either separately/individually and/or in combination/collectively: control circuitry 401, one or more communication interfaces 402, one or more power supply units 403, one or more I/O components 404, and/or one or more mobilization components 405 (e.g., casters or other types of wheels). In some embodiments, the control system 150 can comprise a housing/enclosure configured and/or dimensioned to house or contain at least part of one or more of the components of the control system 150. In this example, the control system 150 is illustrated as a cart-based system that is movable with the one or more mobilization components 405. In some cases, after reaching the appropriate position, the one or more mobilization components 405 can be immobilized using wheel locks to hold the control system 150 in place. However, the control system 150 can be implemented as a stationary system, integrated into another system/device, and so on.

The various components of the control system 150 can be electrically and/or communicatively coupled using certain connectivity circuitry/devices/features, which may or may not be part of control circuitry. For example, the connectivity feature(s) can include one or more printed circuit boards configured to facilitate mounting and/or interconnectivity of at least some of the various components/circuitry of the control system 150. In some embodiments, two or more of the components of the control system 150 can be electrically and/or communicatively coupled to each other.

The one or more communication interfaces 402 can be configured to communicate with one or more devices/sensors/systems. For example, the one or more communication interfaces 402 can send/receive data in a wireless and/or wired manner over a network. In some embodiments, the one or more communication interfaces 402 can implement a wireless technology, such as Bluetooth, Wi-Fi, near field communication (NFC), or the like.

The one or more power supply units 403 can be configured to manage and/or provide power for the control system 150 (and/or the robotic system 110/fluid management system 170, in some cases). In some embodiments, the one or more power supply units 403 include one or more batteries, such as a lithium-based battery, a lead-acid battery, an alkaline battery, and/or another type of battery. That is, the one or more power supply units 403 can comprise one or more devices and/or circuitry configured to provide a source of power and/or provide power management functionality. Moreover, in some embodiments the one or more power supply units 403 include a mains power connector that is configured to couple to an alternating current (AC) or direct current (DC) mains power source.

The one or more I/O components/devices 404 can include a variety of components to receive input and/or provide output, such as to interface with a user to assist in performing a medical procedure. The one or more I/O components 404 can be configured to receive touch, speech, gesture, or any other type of input. In examples, the one or more I/O components 404 can be used to provide input regarding control of a device/system, such as to control the robotic system 110, navigate a scope/catheter or other medical instrument attached to the robotic system 110 and/or deployed through the scope, control the table 180, control a fluoroscopy device, and so on. For example, a physician (not illustrated) can provide input via the I/O component(s) 404 and, in response, the control system 150 can send control signals to the robotic system 110 to manipulate a medical instrument. In examples, the physician can use the same I/O device to control multiple medical instruments (e.g., switch control between the instruments).

As shown, the one or more I/O components 404 can include the one or more displays 156 (sometimes referred to as “the one or more display devices 156”) configured to display data. The one or more displays 156 can include one or more liquid-crystal displays (LCD), light-emitting diode (LED) displays, organic LED displays, plasma displays, electronic paper displays, and/or any other type(s) of technology. In some embodiments, the one or more displays 156 include one or more touchscreens configured to receive input and/or display data. Further, the one or more I/O components 404 can include one or more I/O devices/controls 406, which can include a touch pad, controller (e.g., hand-held controller, video-game-type controller, finger-based controls that enable finger-like movement, etc.), mouse, keyboard, wearable device (e.g., optical head-mounted display), virtual or augmented reality device (e.g., head-mounted display), foot panel (e.g., buttons at the user's feet), etc. Additionally, the one or more I/O components 404 can include one or more speakers configured to output sounds based on audio signals and/or one or more microphones configured to receive sounds and generate audio signals. In some embodiments, the one or more I/O components 404 include or are implemented as a console.

In some embodiments, the one or more I/O components 404 can output information related to a procedure. For example, the control system 150 can receive real-time images that are captured by a scope and display the real-time images and/or visual/image representations of the real-time images via the display(s) 156. The display(s) 156 can present an interface(s), which can include image data from the scope and/or another medical instrument. Additionally, or alternatively, the control system 150 can receive signals (e.g., analog, digital, electrical, acoustic/sonic, pneumatic, tactile, hydraulic, etc.) from a medical monitor and/or a sensor associated with a patient, and the display(s) 156 can present information regarding the health or environment of the patient. Such information can include information that is displayed via a medical monitor including, for example, a heart rate (e.g., ECG, FIRV, etc.), blood pressure/rate, muscle bio-signals (e.g., EMG), body temperature, blood oxygen saturation (e.g., SpO₂), CO₂, brainwaves (e.g., EEG), environmental and/or local or core body temperature, and so on.

In some embodiments, the control system 150 can be coupled to the robotic system 110, a table 180 or another table, and/or a medical instrument, through one or more cables or connections (not shown). In some implementations, support functionality from the control system 150 can be provided through a single cable, simplifying and de-cluttering an operating room. In other implementations, specific functionality can be coupled in separate cabling and connections. For example, while power can be provided through a single power cable, the support for controls, optics, fluidics, and/or navigation can be provided through a separate cable.

The robotic system 110 generally includes an elongate support structure 410 (also referred to as a “column”), a robotic system base 411, and a console 412 at the top of the column 410. The column 410 can include one or more carriages 413 (also referred to as “the arm support 413”) for supporting the deployment of one or more the robotic arms 112. The carriage 413 can include individually configurable arm mounts that rotate along a perpendicular axis to adjust the base of the robotic arms 112 for positioning relative to a patient. The carriage 413 also includes a carriage interface 414 that allows the carriage 413 to vertically translate along the column 410. The carriage interface 414 can be connected to the column 410 through slots, such as slot 415, that are positioned on opposite sides of the column 410 to guide the vertical translation of the carriage 413. The slot 415 can include a vertical translation interface to position and/or hold the carriage 413 at various vertical heights relative to the base 411. Vertical translation of the carriage 413 allows the robotic system 110 to adjust the reach of the robotic arms 112 to meet a variety of table heights, patient sizes, physician preferences. etc. Similarly, the individually configurable arm mounts on the carriage 413 allow a robotic arm base 416 of the robotic arms 112 to be angled in a variety of configurations. The column 410 can internally comprise mechanisms, such as gears and/or motors, that are designed to use a vertically aligned lead screw to translate the carriage 413 in a mechanized fashion in response to control signals generated in response to user inputs, such as inputs from an I/O device(s).

The base 411 can balance the weight of the column 410, the carriage 413, and/or robotic arms 112 over a surface, such as the floor. Accordingly, the base 411 can house heavier components, such as one or more electronics, motors, power supply, etc., as well as components that enable movement and/or immobilize the robotic system 110. For example, the base 411 can include rollable wheels 417 (also referred to as “the casters 417” or “the mobilization components 417”) that allow for the robotic system 110 to move around the room for a procedure. After reaching an appropriate position, the casters 417 can be immobilized using wheel locks to hold the robotic system 110 in place during the procedure. As shown, the robotic system 110 also includes a handle 418 to assist with maneuvering and/or stabilizing the robotic system 110. In this example, the robotic system 110 is illustrated as a cart-based system that is movable. However, the robotic system 110 can be implemented as a stationary system, integrated into a table, and so on.

The robotic arms 112 can generally comprise robotic the arm bases 416 and end effectors 419, separated by a series of linkages 420 (also referred to as “arm segments 420”) that are connected by a series of joints 421. Each joint 421 can comprise an independent actuator and each actuator can comprise an independently controllable motor. Each independently controllable joint 421 represents an independent degree of freedom available to the robotic arm 112. For example, each of the arms 112 can have seven joints, and thus, provide seven degrees of freedom. However, any number of joints can be implemented with any degrees of freedom. In examples, a multitude of joints can result in a multitude of degrees of freedom, allowing for “redundant” degrees of freedom. Redundant degrees of freedom allow the robotic arms 112 to position their respective end effectors 419 at a specific position, orientation, and/or trajectory in space using different linkage positions and/or joint angles. In some embodiments, the end effectors 419 can be configured to engage with and/or control a medical instrument, a device, an object, and so on. The freedom of movement of the arms 112 can allow the robotic system 110 to position and/or direct a medical instrument from a desired point in space and/or allow a physician to move the arms 112 into a clinically advantageous position away from the patient to create access, while avoiding arm collisions.

The end effector 419 of each of the robotic arms 112 can comprise an instrument device manipulator (IDM). In some embodiments, the IDM can be removed and replaced with a different type of IDM. For example, a first type of IDM can manipulate an endoscope, a second type of IDM can manipulate a catheter, a third type of IDM can hold an EM field generator, and so on. However, the same IDM can be used. In some instances, an IDM can include connectors to transfer pneumatic pressure, electrical power, electrical signals, and/or optical signals to/from the robotic arm 112. The IDMs may be configured to manipulate medical instruments using techniques including, for example, direct drives, harmonic drives, geared drives, belts/pulleys, magnetic drives, and the like. In some embodiments, the IDMs can be attached to respective ones of the robotic arms 112, wherein the robotic arms 112 are configured to insert or retract the respective coupled medical instruments into or out of the treatment site.

In some embodiments, the robotic arms 112 can be configured to control a position, orientation, and/or articulation of a medical instrument (e.g., a sheath and/or a leader of a scope) attached thereto. For example, the robotic arms 112 can be configured/configurable to manipulate a scope/catheter using elongate movement members. The elongate movement members can include one or more pull wires, cables, fibers, and/or flexible shafts. To illustrate, the robotic arms 112 can be configured to actuate multiple pull wires of the scope/catheter to deflect the tip of the scope/catheter. Pull wires can include any suitable or desirable materials, such as metallic and/or non-metallic materials such as stainless steel, Kevlar, tungsten, carbon fiber, and the like. In some embodiments, the scope/catheter is configured to exhibit nonlinear behavior in response to forces applied by the elongate movement members. The nonlinear behavior can be based on stiffness and/or compressibility of the scope/catheter, as well as variability in slack or stiffness between different elongate movement members.

As shown, the console 412 is positioned at the upper end of column 410 of the robotic system 110. The console 412 can include a display(s) to provide a user interface for receiving user input and/or providing output (e.g., a dual-purpose device, such as a touchscreen), such as to provide a physician/user with pre-operative data, intra-operative data, information to configure the robotic system 110, and so on. Potential pre-operative data can include pre-operative plans, navigation and mapping data derived from pre-operative computerized tomography (CT) scans, and/or notes from pre-operative patient interviews. Intra-operative data can include optical information provided from a tool, sensor and/or coordinate information from sensors, as well as vital patient statistics, such as respiration, heart rate, and/or pulse. The console 412 can be positioned and tilted to allow a physician to access the console 412 from the side of the column 410 opposite arm base 416. From this position, the physician may view the console 412, robotic arms 112, and patient while operating the console 412 from behind the robotic system 110.

The robotic system 110 can also include control circuitry 422, one or more communication interfaces 423, one or more power supply units 424, one or more input/output components 425, and one or more actuators/hardware 426. The one or more communication interfaces 423 can be configured to communicate with one or more device/sensors/systems. For example, the one or more communication interfaces 423 can send/receive data in a wireless and/or wired manner over a network.

The one or more power supply units 424 can be configured to manage and/or provide power for the robotic system 110. In some embodiments, the one or more power supply units 424 include one or more batteries, such as a lithium-based battery, a lead-acid battery, an alkaline battery, and/or another type of battery. That is, the one or more power supply units 424 can comprise one or more devices and/or circuitry configured to provide a source of power and/or provide power management functionality. Moreover, in some embodiments the one or more power supply units 424 include a mains power connector that is configured to couple to an alternating current (AC) or direct current (DC) mains power source. Further, in some embodiments, the one or more power supply units 424 include a connector that is configured to couple to the control system 150 to receive power from the control system 150.

The one or more I/O components/devices 425 can be configured to receive input and/or provide output, such as to interface with a user. The one or more I/O components 425 can be configured to receive touch, speech, gesture, or any other type of input. In examples, the one or more I/O components 425 can be used to provide input regarding control of a device/system, such as to control/configure the robotic system 110. The one or more I/O components 425 can include the one or more displays configured to display data. The one or more displays can include one or more liquid-crystal displays (LCD), light-emitting diode (LED) displays, organic LED displays, plasma displays, electronic paper displays, and/or any other type(s) of technology. In some embodiments, the one or more displays include one or more touchscreens configured to receive input and/or display data. Further, the one or more I/O components 425 can include a touch pad, controller, mouse, keyboard, wearable device (e.g., optical head-mounted display), virtual or augmented reality device (e.g., head-mounted display), etc. Additionally, the one or more I/O components 425 can include one or more speakers configured to output sounds based on audio signals and/or one or more microphones configured to receive sounds and generate audio signals. In some embodiments, the one or more I/O components 425 include or are implemented as the console 412. Further, the one or more I/O components 425 can include one or more buttons that can be physically pressed, such as a button on a distal end of a robotic arm 112 (which can enable/disable an admittance control mode of the robotic arm 112 for manual manipulation/movement of the robotic arm 112).

The one or more actuators/hardware 426 can be configured to facilitate movement of the robotic arms 112. Each actuator 426 can comprise a motor, which can be implemented in a joint or elsewhere within a robotic arm 112 to facilitate movement of the joint and/or a connected arm segment/linkage. In some embodiments, a user can manually manipulate a robotic arm 112 without using electronic user controls. For example, during setup in a surgical operating room or at any point during a procedure, a user may select a button on a distal end of a robotic arm 112 to enable an admittance control mode and then manually move the robotic arm 112 to a particular orientation/position.

The various components of the robotic system 110 can be electrically and/or communicatively coupled using certain connectivity circuitry/devices/features, which may or may not be part of the control circuitry 422. For example, the connectivity feature(s) can include one or more printed circuit boards configured to facilitate mounting and/or interconnectivity of at least some of the various components/circuitry of the robotic system 110. In some embodiments, two or more of the components of the robotic system 110 can be electrically and/or communicatively coupled to each other.

The robotic fluid management system 170 can include control circuitry 430, one or more communication interfaces 432, one or more power supply units 433, one or more input/output components 434, one or more pumps 435, one or more vacuums 436, and an irrigation fluid source 437. The one or more communication interfaces 432 can be configured to communicate with one or more device/sensors/systems. For example, the one or more communication interfaces 432 can send/receive data in a wireless and/or wired manner over a network.

The one or more power supply units 433 can be configured to manage and/or provide power for the fluid management system 170. In some embodiments, the one or more power supply units 433 include one or more batteries, such as a lithium-based battery, a lead-acid battery, an alkaline battery, and/or another type of battery. That is, the one or more power supply units 433 can comprise one or more devices and/or circuitry configured to provide a source of power and/or provide power management functionality. Moreover, in some embodiments the one or more power supply units 433 include a mains power connector that is configured to couple to an alternating current (AC) or direct current (DC) mains power source. Further, in some embodiments, the one or more power supply units 433 include a connector that is configured to couple to the control system 150 to receive power from the control system 150.

The one or more I/O components/devices 434 can be configured to receive input and/or provide output, such as to interface with a user. The one or more I/O components 434 can be configured to receive touch, speech, gesture, or any other type of input. The one or more I/O components 434 can include a display, a touch pad, controller, mouse, keyboard, wearable device (e.g., optical head-mounted display), virtual or augmented reality device (e.g., head-mounted display), speaker, microphone, etc. Further, the one or more I/O components 434 can include one or more buttons that can be physically pressed.

The fluid management system 170 can be configured to control the pump(s) 435 and/or the vacuum(s) 436 to provide irrigation/aspiration. For example, a medical instrument may be attached to the pump(s) 435/vacuum 436 to provide irrigation/aspiration to a target site via medical instrument. In examples, the fluid management system 170 can include one or more flow meters, valve controls, and/or other fluid-/flow-control components (e.g., sensor devices, such as pressure sensors) in order to provide controlled irrigation and/or aspiration/suction capabilities for a medical instrument. In some embodiments, the control system 150 and/or the robotic system 110 can generate and provide one or more signals to the fluid management system 170 to control irrigation/aspiration.

The pump(s) 435 can be attached to an irrigation fluid source 437, which can include the fluid bag(s)/container(s) 171 and/or a fluid line(s)/connector(s) 438 to connect to a medical instrument(s). The pump(s) 435 can pump irrigation fluid (e.g., saline solution) through one or more medical instruments and into a treatment site. In some examples, the pump(s) 435 is a peristaltic pump(s). In some embodiments, the pump(s) 435 can be replaced with a vacuum that is configured to apply a vacuum pressure to draw the irrigation fluid from the irrigation fluid source 437 and out through the respective coupled medical instrument. Although FIG. 4 includes the pump(s) 435, in some embodiments, irrigation fluid flow is achieved without the use of pumps, wherein such flow is driven primarily by gravitational force.

The vacuum(s) 436 can be configured to facilitate fluid aspiration. For example, the vacuum(s) 436 can be configured to apply a negative pressure to draw fluid out of a treatment site. The vacuum(s) 436 may be connected to a collection container into which withdrawn fluid is collected. In some examples, aspiration suction may be facilitated by one or more pumps rather than a vacuum. Furthermore, in some embodiments, aspiration is primarily passive, rather than through active suction. Therefore, it should be understood that embodiments of the present disclosure may not include vacuum components.

As referenced above, the systems 150, 110, and 170 can include the control circuitry 401, 422, and 430, respectively, configured to perform certain functionality described herein. The term “control circuitry” can refer to any collection of one or more processors, processing circuitry, processing modules/units, chips, dies (e.g., semiconductor dies including one or more active and/or passive devices and/or connectivity circuitry), microprocessors, micro-controllers, digital signal processors, microcomputers, central processing units, graphics processing units, field programmable gate arrays, application specific integrated circuits, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry can further comprise one or more, storage devices, which can be embodied in a single memory device, a plurality of memory devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware state machine (and/or implements a software state machine), analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Although control circuitry is illustrated as a separate component from other components of the control system 150/robotic system 110/fluid management system 170, any or all of the other components of the control system 150/robotic system 110/fluid management system 170 can be embodied at least in part in the control circuitry. For instance, control circuitry can include various devices (active and/or passive), semiconductor materials and/or areas, layers, regions, and/or portions thereof, conductors, leads, vias, connections, and/or the like, wherein one or more of the other components of the control system 150/robotic system 110/fluid management system 170 and/or portion(s) thereof can be formed and/or embodied at least in part in/by such circuitry components/devices.

Further, although not illustrated in FIG. 4, one or more of the control system 150, the robotic system 110, and/or the fluid management system 170 can each include data storage/memory configured to store data/instructions. For example, data storage/memory can store instructions that are executable by control circuitry to perform certain functionality/operations. The term “memory” can refer to any suitable or desirable type of computer-readable media. For example, one or more computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data. One or more computer-readable media that can be implemented in accordance with embodiments of the present disclosure includes, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

In some instances, the control system 150 and/or the robotic system 110 is configured to implement one or more localization techniques to determine/track an orientation/position of an object/medical instrument. For example, the one or more localization techniques can process input data to generate position/orientation data for a medical instrument. Position/orientation data of an object/medical instrument can indicate a position/orientation of the object/medical instrument relative to a frame of reference. The frame of reference can be a frame of reference relative to anatomy of a patient, a known object (e.g., an EM field generator, system, etc.), a coordinate system/space, and so on. In some implementations, position/orientation data can indicate a position/orientation of a distal end of a medical instrument (and/or proximal end, in some cases). For example, position/orientation data for a scope can indicate a position and orientation of a distal end of the scope, including an amount of roll of the distal end of the scope. A position and orientation of an object can be referred to as a pose of the object.

Example input data that can be used to generate position/orientation data for an object/medical instrument can include: sensor data from a sensor associated with a medical instrument (e.g., EM field sensor data, vision/image data captured by an imaging device/depth sensor/radar device on the medical instrument, accelerometer data from an accelerometer on the medical instrument, gyroscope data from a gyroscope on the medical instrument, satellite-based positioning data from a satellite-based sensor (a global positioning system (GPS), for example), and so on); feedback data from a robotic arm/component (also referred to as “kinematics data”) (e.g., data indicating how a robotic arm/component moved/actuated); robotic command data for a robotic arm/component (e.g., a control signal sent to the robotic system 110/robotic arm 112 to control movement of the robotic arm 112/medical instrument); shape sensing data from a shape sensing fiber (which can provide information regarding a location/shape of a medical instrument); model data regarding anatomy of a patient (e.g., a model of an interior/exterior portion of anatomy of the patient); position data of a patient (e.g., data indicating how the patient is positioned on a table); pre-operative data; etc.

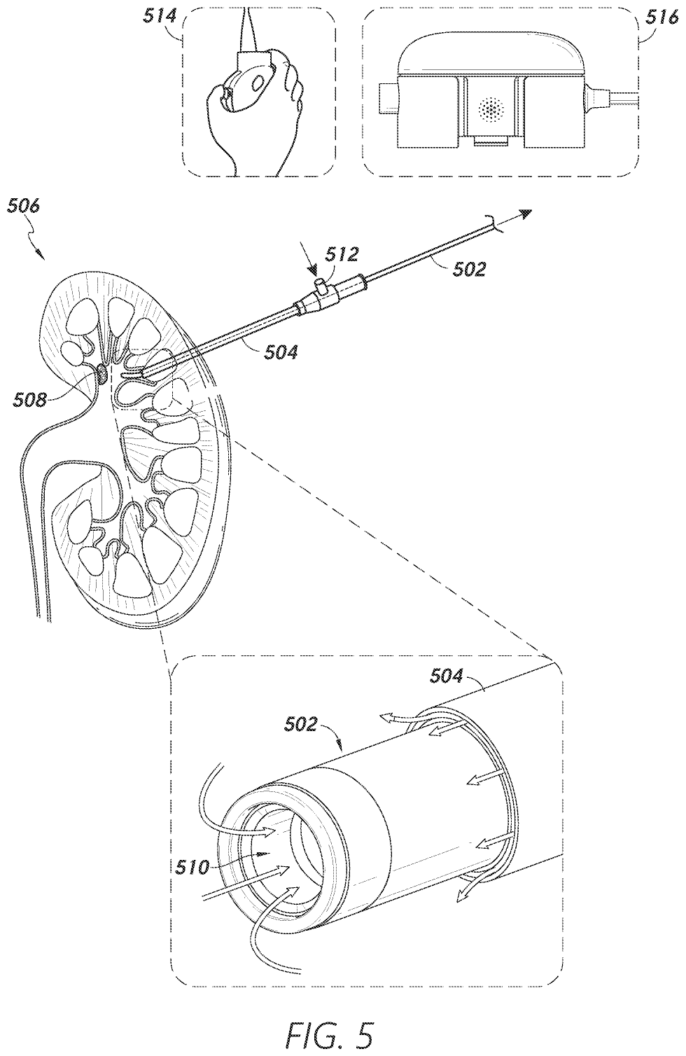

FIG. 5 illustrates an example catheter 502 and a percutaneous-access device 504 disposed at least partly in a kidney 506 of a patient in accordance with one or more embodiments. The catheter 502 and percutaneous-access device 504 may be representative any of the catheters and percutaneous-access devices discussed herein. In this example, the instruments 502, 504 are illustrated in the context of a urology procedure to treat/remove a kidney stone 508 from the kidney 506. However, the instruments 502, 504 can be used in other types of procedures. As noted above, urology procedures and/or other types of procedures can be implemented manually at least in part and/or can be performed using robotic technologies at least in part.

The catheter 502 can be configured to be articulated, such as with respect to at least a distal end/tip of the catheter 502. For instance, the distal end portion/tip of the catheter 502 can be deflected in a variety of directions. In examples, the catheter 502 can be configured to move with two degrees of freedom (2-DOF) (e.g., two of x, y, z, yaw, pitch, or roll movement). To illustrate, the distal end portion of the catheter 502 can be configured to move right/left or up/down (e.g., x, y, or z movement) and also move to insert/retract the catheter 502 (e.g., translate along the x, y, or z axis). In other examples, the catheter 502 can be configured to move with 3-DOF (e.g., three of x, y, z, yaw, pitch, or roll movement). To illustrate, the distal end portion of the catheter 502 can be configured to move right/left and up/down (e.g., two of x, y, or z movement) and also move to insert/retract the catheter 502. However, the catheter 502 can also be configured to move with 4-DOF (e.g., x, y, z, and pitch/yaw/roll movement), 6-DOF (e.g., x, y, z, pitch, yaw, and roll movement), and so on. In some embodiments, such as when the catheter 502 is implemented with a robotically-controllable handle, the catheter 502 is not configured for roll movements. However, the catheter 502 can be configured for roll and/or other types of movement in some cases, such as when the catheter 502 is configured with a manually-controllable handle or some robotically-controllable cases.

As shown, the catheter 502 can be implemented with the percutaneous-access device 504 to provide aspiration/irrigation to the kidney 506. The percutaneous-access device 504 may include one or more sheaths and/or shafts through which instruments (e.g., the catheter 502) and/or fluids may access the target anatomy in which the distal end of the device 504 is disposed. In some embodiments, active aspiration/suction may be drawn through a lumen 510 of the catheter 502 to a proximal end of the catheter 502 (e.g., a handle of the catheter 502). Further, in some embodiments, irrigation can be provided via the percutaneous-access device 504, such as between concentric sheaths. For example, a fluid management system (not illustrated) can be connected to an irrigation port 512 port to provide irrigation to the percutaneous-access device 504, which travels down the percutaneous-access device 504 to the target site. FIG. 5 illustrates an example of the flow of aspiration fluid into the lumen 510 of the catheter 502 and the flow of irrigation fluid from the percutaneous-access device 504. In some embodiments, a passive aspiration outflow channel may be formed in the space between the outer wall of the catheter 502 and an inner wall/sheath of the percutaneous-access device/assembly 504. When the catheter 502 is disposed within the percutaneous-access device 504, the catheter 502 and the shaft(s)/sheath(s) of the percutaneous-access device 504 may be generally concentric. The catheter 502 and the percutaneous access device 504 may have generally circular cross-sectional shapes over at least portions thereof.

The catheter 502 may be controllable in any suitable or desirable way, either based on manual control and/or robotic control. In FIG. 5, handles/bases 514, 516 provide examples that may be used to control the catheter 502. The handle 514 illustrates a hand-held/manual handle that is configured to be manipulated by a physician/user to control movement of the catheter 502. Meanwhile, the handle 516 illustrates a robotically controllable handle that is configured to be manipulated by a robotic arm, such as an end effector of a robotic arm, to control movement of the catheter 502. Example robotically-controllable and manually-controllable catheters are discussed in further detail below. By implementing an articulable catheter, the techniques/structures can allow various positions within the patient to be reached in a manner that prevents/minimizes damage to the anatomy of the patient. For example, a physician can navigate the distal portion of the catheter 502 to reach a particular cavity in the kidney 506 (e.g., calyx) where a kidney stone is located, without repositioning the rest of the shaft of the catheter 502 and/or the percutaneous-access device 504.

In embodiments, the catheter 502 is free of an imaging device. That is, the catheter 502 is implemented without an imaging device/camera on a distal end to capture image data of an internal anatomy of the patient. However, in other embodiments the catheter 502 can include an imaging device(s), such as on the tip of the catheter 502. Further, in embodiments, the catheter 502 is implemented without a position sensor (i.e., does not include a position sensor). However, the catheter 502 can be implemented with a position sensor in some cases, such as on a distal end of the catheter 502.

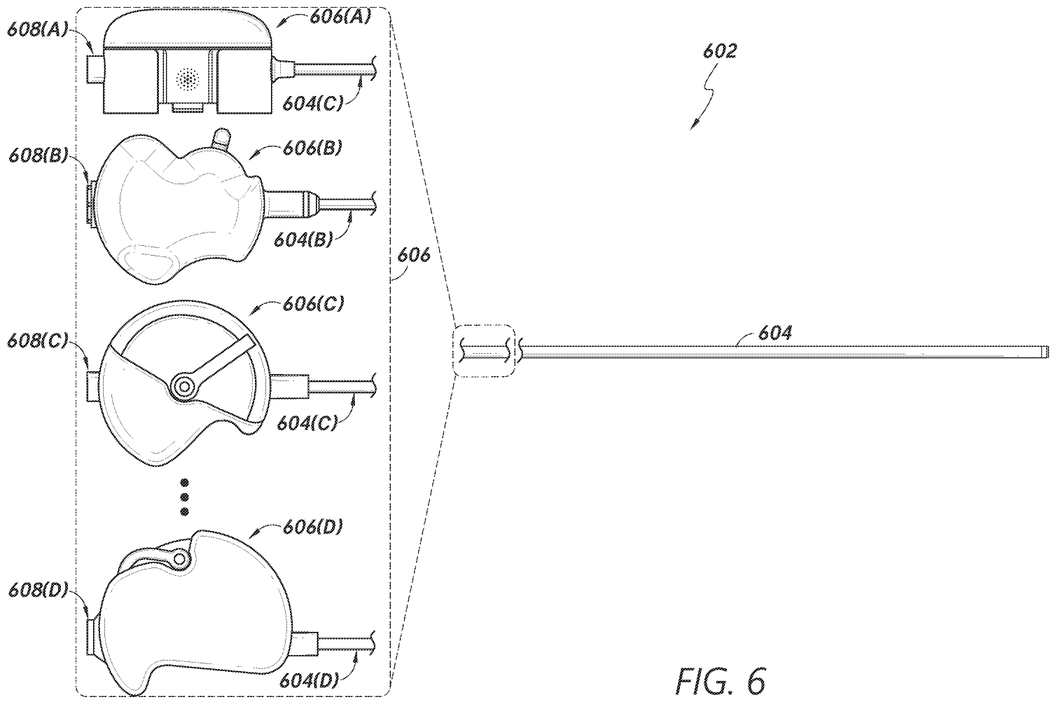

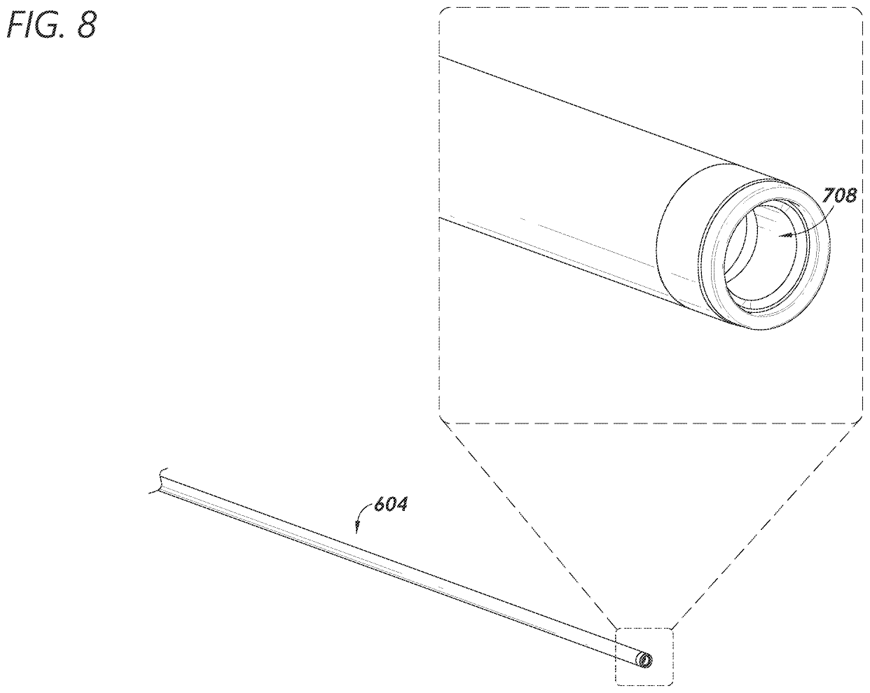

FIGS. 6-13 illustrates example features of a robotically/manually controllable catheter 602 in accordance with one or more embodiments of the present disclosure. The features of the catheter 602 may be implemented in the context of one or more of the catheters discussed herein. The catheter 602 includes an elongate shaft 604 connected to a handle/base 606 (also referred to as the “instrument base 606”) that is configured to control actuation of at least a portion of the elongate shaft 604. As shown in FIG. 6, the handle 606 can be implemented as a robotically controllable handle (e.g., the handle 606(A)) configured to couple to a robotic arm and/or a manually controllable handle (e.g., the handles 606(B), 606(C), and 606(D)) configured to be held/manipulated by a user. In some embodiments, the elongate shaft 604 can extend through the handle 606 to a port 608 of the handle 606, which can be connected to a fluid management system and/or another system to facilitate aspiration, irrigation, deployment of an instrument through a working channel of the catheter 602, and so on. Although certain handles are discussed in the context of being implemented in a manually-controllable catheter or robotically-controllable catheter, such catheters can be implemented in other contexts. For example, a manually-controllable catheter can include robotic components to be implemented as a robotically-controllable catheter (e.g., secondary use as a robotic catheter), and/or a robotically-controllable catheter can include manual component to be implemented as a manually-controllable catheter (e.g., secondary use as a manual catheter). As such, in some cases, a catheter is configured for both manual and robotic manipulation.

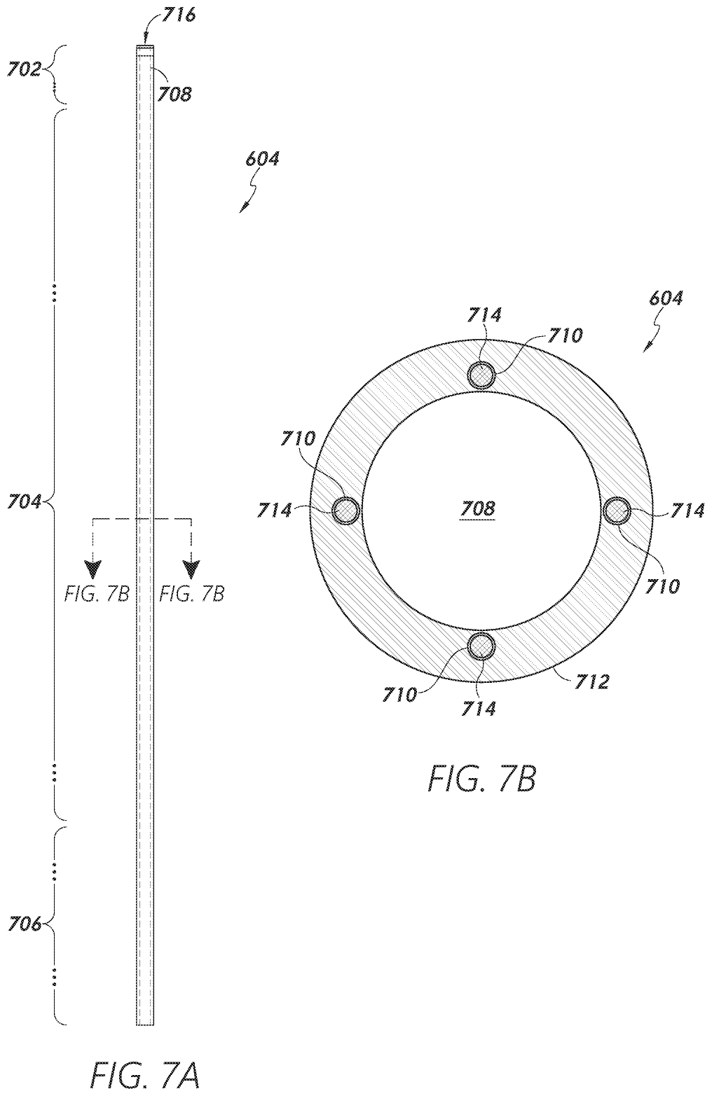

As shown in FIGS. 7A and 7B (and other figures), the shaft 604 can include a distal/tip section/portion 702 (sometimes referred to as “the distal end portion 702”), a middle/medial section/portion 704, a proximal section/portion 706 (sometimes referred to as “the proximal end portion 706”), and/or a lumen 708 that extends through at least a portion of the shaft 604. For example, the lumen 708 can extend through an entirety of the shaft 604 from the distal section 702 (that may be positioned at a target site in a patient) to the proximal section 706 (that may be connected to the port 608 of the handle 606). However, the lumen 708 can extend another distance through the catheter 602. In examples, the lumen 708 can be referred to as a working channel. The distal section 702, the middle section 704, and/or the proximal section 706 can each be implemented with any longitudinal length. The terms distal, middle/medial, proximal, and/or other terms are used to describe a position of a feature relative to another feature. For example, a proximal feature of the catheter 602 can refer to a feature that is farthest from a target or anatomical site (e.g., during use/a procedure), whereas a distal feature of the catheter 602 can refer to a feature that is closest to the target or anatomical site.

The distal section 702 of the shaft 604 can include a filter/containment structure/feature 716 (also referred to as “the tip structure 716”) configured to prevent certain objects from entering into the rest of the shaft 604 and/or configured to contain an object at a distal end of the shaft 604, such as when aspirating through the shaft 604. For example, in the context of a urological procedure, the distal portion 702 of the catheter 602 can be positioned at a target site and used to aspirate one or more kidney stone fragments from a kidney. Here, the tip structure 716 can be configured to hold the kidney stone while the stone is being fragmented into pieces, such as by an instrument deployed from another device at the target site. The tip structure 716 can also prevent fragments that are larger than a particular size from being sucked into the rest of the shaft 604, which could clog the shaft 604 and impede/stop aspiration flow. Although the tip structure 716 is illustrated as a separate component from the rest of the shaft 604 in many examples (e.g., removably coupled to the rest of the shaft 604), the tip structure 716 can be integral with rest of the shaft 604 or implemented in other manners. Example features of the tip structure 716 are discussed in further detail below.

In some embodiments, at least a portion of the shaft 604 can be formed of various materials, such as plastics, rubbers, vertebrae links, metal or plastic braids/coils, and so on, such that at least a portion of the shaft 604 is flexible for articulation. In some embodiments, the shaft 604 includes reinforcement material (e.g., braided) to strengthen and/or facilitate flexibility of the shaft 604. For example, the shaft 604 can include braid reinforcement for hoop strength and or to prevent kinking of the shaft 604 when the shaft 604 is navigated within the anatomy of a patient. Further, in some embodiments, the shaft 604 includes multiple layers of material that are implemented in a variety of configurations to facilitate the features of the shaft 604 discussed herein. In some cases, the tip structure 716 is formed of a different material than the rest of the shaft 604. For example, the tip structure 716 can be implemented with a material that avoids degradation in certain contexts, such as catastrophic degradation. The tip structure 716 can be implemented with stainless steel (or other types of steel), titanium, tungsten, aluminum alloy, iron alloy, steel alloy, titanium alloy, tungsten alloy, and/or other materials (which may have relatively high melting points above a threshold) that can generally maintain its structure when laser beams inadvertently and/or occasionally contact the tip structure 716. However, the tip structure 716 and/or any other portion of the shaft 604 can be implemented with other materials. In some instances, the tip structure 716 includes certain materials that can have reduced degradation in certain contexts, such as when contacted by a laser. For example, the tip structure 716 can be formed of a material that has a fracture toughness greater than or equal to 2 MPa·m^{1/2}. However, other fracture toughness values/ranges can be implemented.

The shaft 604 can include one or more lumens 710 (also referred to as “the one or more wire lumens 710”) disposed in a wall 712 of the shaft 604, such as an outer wall, as shown in the cross-sectional view of FIG. 7B taken along the line shown in FIG. 7A. The one or more lumens 710 can be spaced equidistantly apart around the wall of the shaft 604 or at another location. The catheter 604 can include one or more elongate movement members 714 slidably disposed in the one or more wire lumens 710 (also referred to as “wall lumens 710”). The one or more elongate movement members 714 can include one or more pull wires, cables, fibers, and/or flexible shafts. The one or more elongate movement members 714 can include any suitable or desirable materials, such as metallic and non-metallic materials, including stainless steel, Kevlar, tungsten, carbon fiber, and the like. In some embodiments, the catheter 602 is configured to exhibit nonlinear behavior in response to forces applied by the one or more elongate movement members 714. The nonlinear behavior may be based on stiffness and/or compressibility of the catheter 602, as well as variability in slack or stiffness between different elongate movement members 714. Although a particular number of wire lumens 710 and elongate movement members 714 are illustrated in the figures, any number of lumens and/or elongate movement members can be implemented.

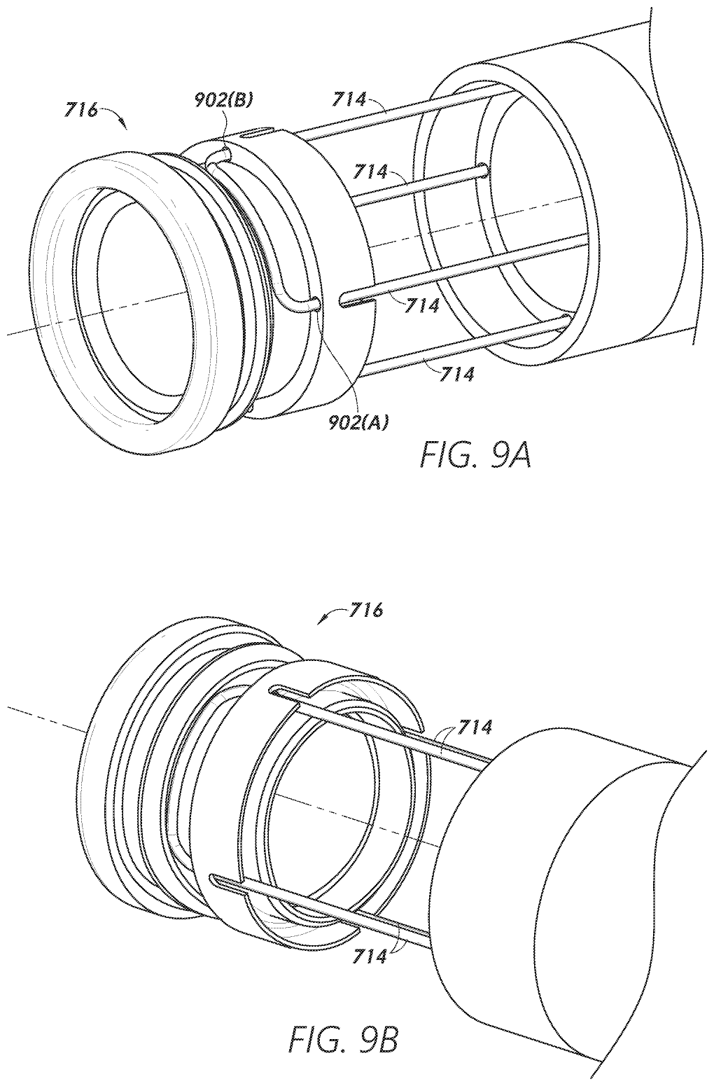

The one or more elongate movement members 714 can be attached/extend to the distal section 702 of the shaft 604, as shown in FIGS. 9, 10, and 13. At a proximal side, the one or more elongate movement members 714 can be coupled to a component(s) of the handle 606 (e.g., an input assembly) that is configured to control articulation of the shaft 604, such as by deflecting the distal section 702 of the shaft 604. The handle 606 can be configured to pull (and/or release tension of) the one or more elongate movement members 714 within the one or more lumens 710 to cause the distal section 702 to deflect from a longitudinal axis.

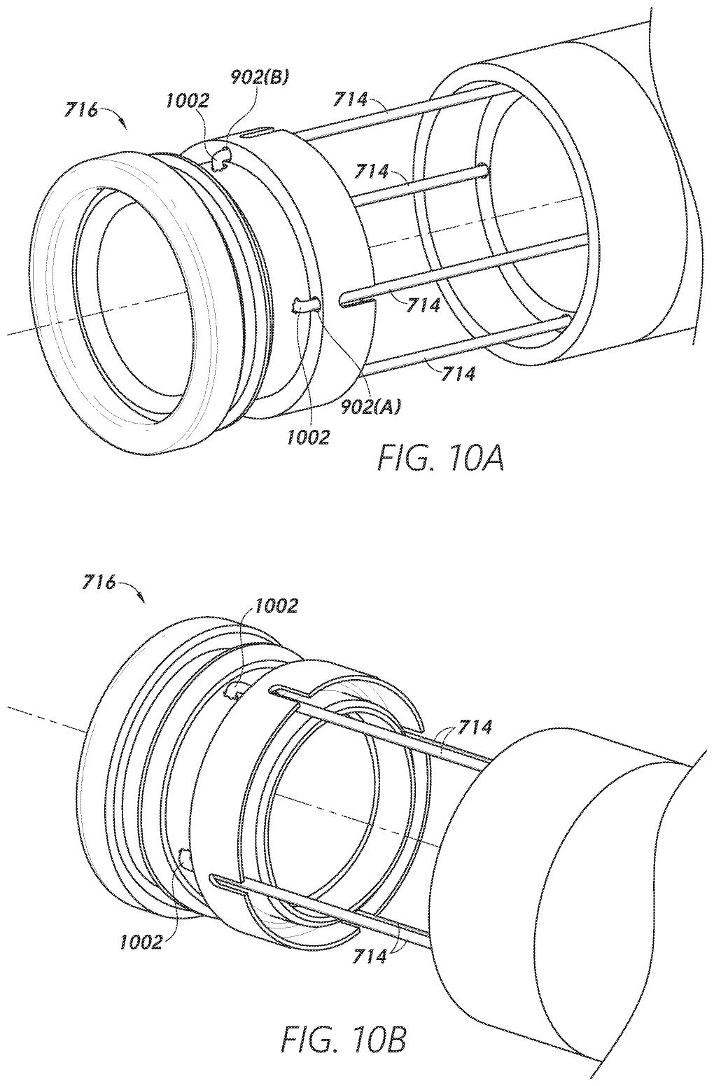

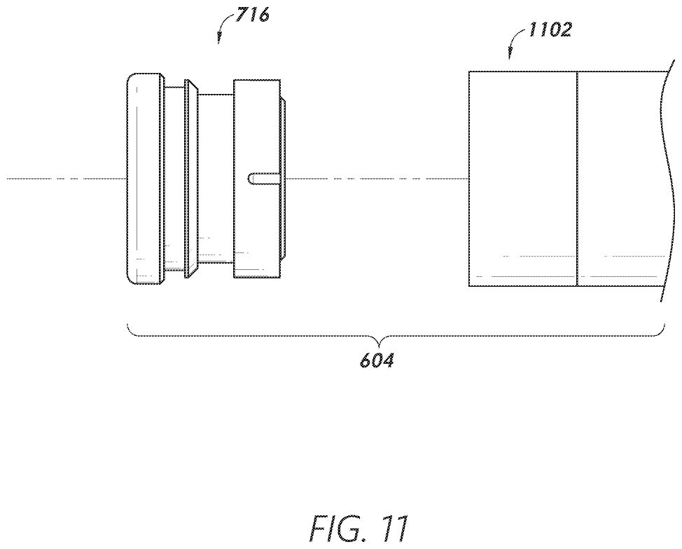

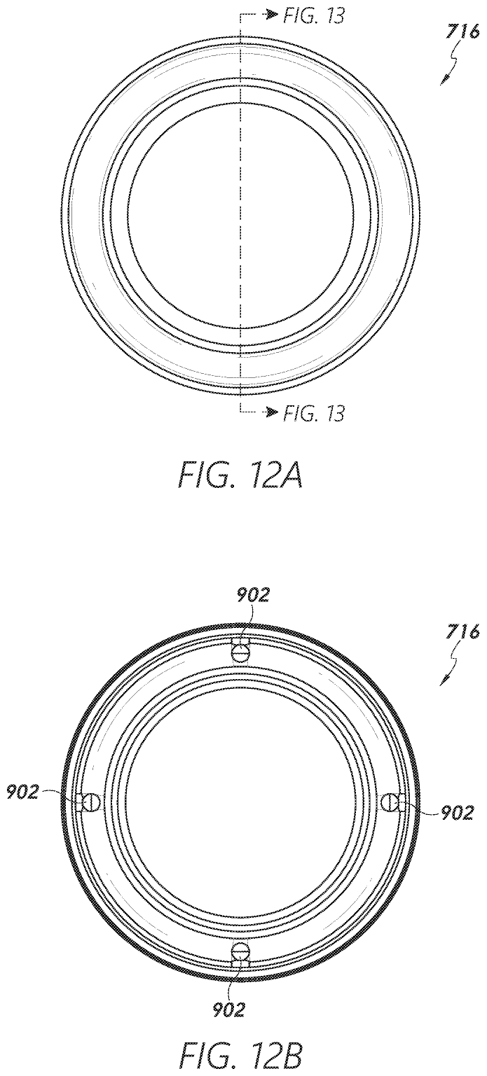

In some examples, as shown in FIGS. 9A and 9B, the one or more elongate movement members 714 attach to the tip structure 716 to articulate the distal portion 702 of the shaft 604. Here, the one or more elongate movement members 714 form a loop-like structure around a portion of the tip structure 716. For example, an elongate movement member 714 extends from the proximal portion 706 of the shaft 604, out a first hole 902(A) in the tip structure 716, extends over a portion of the tip structure 716 to a second hole 902(B), and returns to the proximal portion 706 of the shaft 604 via the second hole 902(B). This can attach the elongate movement member 714 to the tip structure 716. Here, the catheter 602 can generally be configured to move in two directions based on manipulation of the one or more elongate movement members 714 (e.g., up/down or right/left). In other examples, as shown in FIGS. 10A and 10B, the one or more elongate movement members 714 individually attach to the tip structure 716 at anchor points 1002 (which can be implemented in a variety of manners, such as through laser melted ball ends, soldering anchors, etc.). Here, the catheter 602 can be configured to move in four directions based on manipulation of the one or more elongate movement members 714 (e.g., up/down and right/left). In yet other examples, the one or more elongate movement members 714 attach to the shaft 604 in another manner that includes attachment to the tip structure 716 or another section of the distal portion 702. FIG. 11 illustrates the tip structure 716 removed from the rest of the shaft 604 (in a similar manner as shown in FIGS. 9A-9B and but with the one or more elongate movement members 714 removed (e.g., an exploded view without the one or more elongate movement members 714). Meanwhile, FIG. 12A illustrates a front view of the tip structure 716 and FIG. 12B illustrates a rear view of the tip structure 716 with the holes 902 to receive the one or more elongate movement members 714.

In instances where the tip structure 716 is implemented as a separate component from the rest of the shaft 604, the tip structure 716 can be attached to the rest of the shaft 604 with an adhesive, fastener, interlocking mechanism (e.g., tabs, grooves, etc.), and so on. In some embodiments, the shaft 604 includes a ring portion 1102 (as shown in FIG. 11 and elsewhere) to facilitate coupling of the tip structure 716 to the rest of the shaft 604 and/or to cover the tip structure 716 once the tip structure 716 is secured to the rest of the shaft 604. In this example, the shaft 604 (including the tip structure 716) are implemented in a substantially cylindrical form (e.g., having a circular cross-section); however, the shaft 604 can be take in other forms, such as a rectangular/square form or another shape.

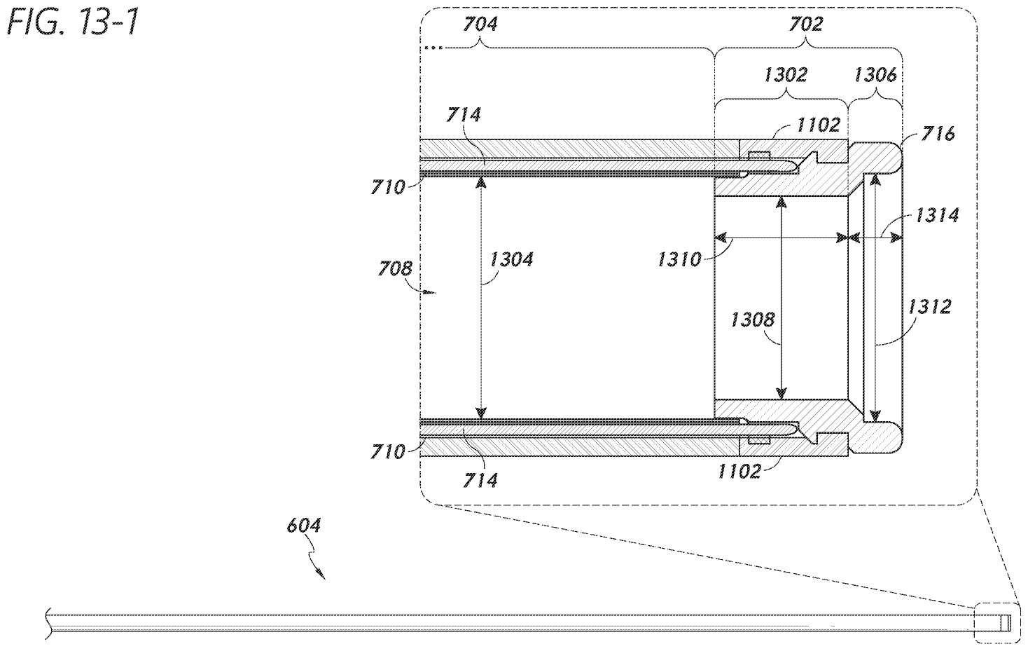

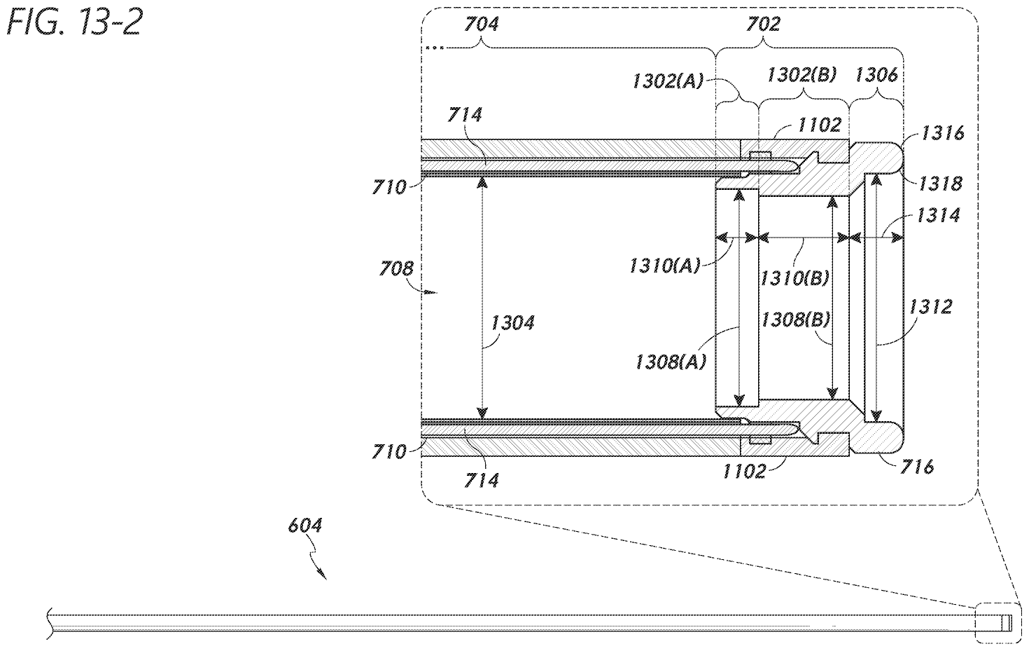

FIGS. 13-1 and 13-2 show two example implementations of the shaft 604 to illustrate various features of the tip structure 716 and the rest of the shaft 604. Each figure illustrates a cross-sectional view of the tip structure 716 taken along the cross-sectional line of FIG. 12A. In particular, FIG. 13-1 illustrates the tip structure 716 having a control section 1302 with a substantially uniform inner diameter, while FIG. 13-2 illustrates the tip structure 716 with the control section 1302 having multiple inner diameters. For ease of discussion, the tip structure 716 may be referred to as the distal portion 702 of the shaft 604. However, it should be understood that the distal portion 702 can extend more or less than the length illustrated. For example, the distal portion 702 of the shaft 604 can extend beyond the tip structure 716 toward a proximal end of the shaft 604.

As shown in the example of FIG. 13-1, an inner diameter of at least a portion of the tip structure 716 can be smaller than an inner diameter 1304 of the middle portion 704 of the shaft 604 to prevent certain objects from entering into the middle portion 704. For example, the tip structure 716 can include the first portion 1302 (i.e., the most proximal portion) (also referred to as “the control section 1302”) and a second portion/section 1306 (i.e., the most distal portion) (also referred to as “the counterbore/countersink section 1306”) adjacent/distal to the control section 1302. Here, the control section 1302 has an inner diameter 1308 that is smaller than the inner diameter 1304 of the middle portion 704. In other words, the inner diameter 1304 is larger than the inner diameter 1308. In some embodiments, a ratio of the inner diameter (ID) 1308 of the control section 1302 to the inner diameter 1304 of the shaft portion 704

$$( i . e . , \frac{ID 1308}{ID 1304} )$$

can be within a range of 0.4 to 0.9; 0.5 to 0.9; 0.4 to 0.8; 0.5 to 0.8; or other ranges. In examples, such ratio can provide an optimum ratio for filtering objects (e.g., stones) while not hindering object removal efficiency.

Further, in some examples, such as that shown in FIG. 13-1, a longitudinal length 1310 of the control section 1302 is less than the inner diameter 1308 of the control section 1302. The length 1310 of the control section 1302 can assist in controlling an orientation of an object entering into the shaft portion 704. However, in other examples, the length 1310 of the control section 1302 is the same as the inner diameter 1308 or greater than the inner diameter 1308. In some illustrations, a ratio of the length 1310 to the inner diameter 1308 (i.e., 1310/1308) can be greater than or equal to 0.4. However, other ratios can be implemented.

By implementing the control section 1302 with one or more of the features discussed herein, the tip structure 716 can prevent objects of a particular size/shape from entering into the rest of the shaft 704. For example, the catheter 602 can be used to aspirate a target site, wherein fluid flows through the lumen 708 of the shaft 604 from the tip structure 716 to the proximal portion of the shaft 604. As the catheter 602 attempts to suck one or more objects into the shaft 604, the control section 1302 can prevent objects that are greater than a particular size and/or having a particular shape from entering into the middle portion 704 of the shaft 604. In examples, the control section 1302 can limit a size of an object in at least two dimensions (e.g., width and height) from traveling into the rest of the shaft 604 to prevent clogging of the shaft 604. Further, the length 1310 of the control section 1302 can be designed to help prevent objects that might otherwise pass through the control section 1302 in a particular orientation and/or due to a shape of the object (e.g., oblong objects) from entering into the rest of the shaft 604.

As also shown in FIG. 13-1, the tip structure 716 includes the countersink/counterbore section 1306 to hold/stabilize an object at the distal end of the shaft 604. In particular, an inner diameter 1312 of the countersink section 1306 is larger than the inner diameter 1308 of the control section 1302. The inner diameter 1312 can be the same size as the inner diameter 1304 of the shaft portion 704 or smaller/larger than the inner diameter 1304 of the shaft portion 704. In any event, the difference in inner diameters of the control section 1302 and the countersink section 1306 can create a feature to hold/stabilize at least a portion of an object. Although a length 1314 of the countersink section 1306 is illustrated as being smaller than a length of the control section 1310 in this example, the length 1314 can be the same or larger than the length 1310 of the control section 1302. In some cases, a length of the tip structure 716 (i.e., the length 1314 and/or the length 1310) is less than the inner diameter 1308 of the control section 1302. However, other lengths can be implemented. As shown, the countersink section 1306 can transition into the control section 1302 with a beveled edge (e.g., a countersunk hole in this example). However, other types of transitions can be implemented, such as a curved edge, counterbore hole, and so on.

As noted above, by implementing the countersink section 1306, the shaft 604 can hold/stabilize an object. For example, the catheter 602 can be used in cooperation with a scope to remove a kidney stone from a kidney of a patient. The scope can deploy an instrument (e.g., laser, ultrasonic fragmenting, etc.) to fragment the kidney stone into pieces that are small enough to be removed via the catheter 602. In many cases, the fragmenting instrument (and/or the irrigation/aspiration provided into the cavity) can cause the kidney stone to move around within the kidney, which can make it difficult to accurately lase/cut the stone, resulting in damage to surrounding anatomy of the patient (e.g., due to the laser/ultrasonic fragmenting/lithotripter inadvertently contacting the patient's anatomy). For instance, the kidney stone can move when contacted by a laser. As such, the countersink section 1306 (and/or aspiration facilitated by the catheter 602) can enable the catheter 602 to hold the kidney stone in place (e.g., within the distal end of the shaft 604) while the stone is fragmented and aspirated out of the kidney.

FIG. 13-2 illustrates the tip structure 716 with the control section 1302 having multiple inner diameters. As shown, the control section 1302 can have two subsections 1302(A) and 1302(B), wherein the first subsection 1302(A) has a larger inner diameter 1308(A) than the inner diameter 1308(B) of the second subsection 1302(B). However, in other cases, the inner diameter 1308(A) can be the same or smaller than the inner diameter 1308(B). In this example, a length 1310(A) of the first subsection 1302(A) is smaller than a length 1310(B) of the second subsection 1302(B). However, the length 1310(A) can be the same or greater than the length 1310(B).

In the examples of FIGS. 13-1 and 13-2, the tip structure 716 includes a substantially rounded distal end. For example, as referenced in FIG. 13-2, the distal end of the tip structure 716 is rounded on an exterior surface/edge 1316 and on an interior surface/edge 1318 (e.g., the tip structure 716 includes a rounded edge profile). This can avoid damaging the anatomy of the patient when the catheter 602 contacts the tissue of the patient during navigation. However, the tip structure 716 can include other forms, such as a rectangular-shaped edge. Further, in many examples, the shaft 604 has a circular cross section; however, the shaft 604 can have other cross-sectional forms, such as rectangular/square forms (e.g., parallelepiped). In such cases, instead of multiple portions/sections of the shaft 604 having different inner diameters, the portions/sections can have different widths, heights, and so on.

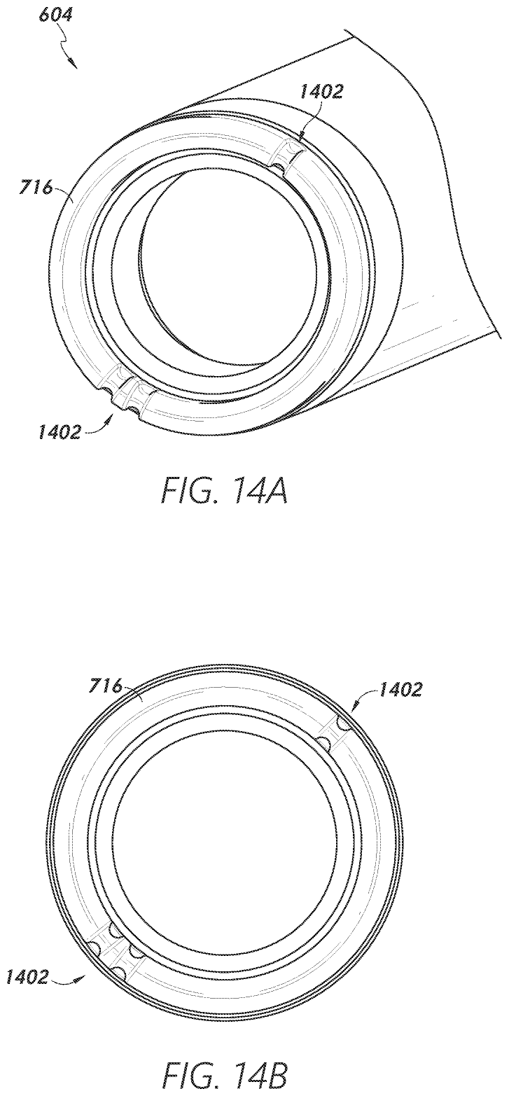

FIGS. 14A and 14B illustrates perspective and front views, respectively, of one or more markings 1402 that can be implemented in some examples on the tip structure 716 of the shaft 604 in accordance with one or more embodiments. In examples, the one or more markings 1402 (also referred to as “orientation marking(s) 1402”) can be used to view an orientation of the distal end of the shaft 604, since the shaft 604 can have a cylindrical form. For example, when viewing the shaft 604 from the perspective of another device, such as from the perspective of a scope that is positioned within proximity to the shaft 604, the one or more markings 1402 can assist a user in identifying an orientation of the distal end of the shaft 604 (e.g., an amount of roll of the shaft 604 relative to the scope and/or relative to the anatomy of the patient). Although various figures are depicted herein without the one or more markings 1402, any of the example shafts/tip structures discussed herein can include the one or more markings 1402.

The one or more markings 1402 can include a deformation(s) (e.g., indentation, hole, notch, flat section, etc.), coloring (e.g., coloring one side of the tip a first color and the other side a different color, color different sides the same color, colored roman numerals, etc.), image(s) (e.g., a number, letter, shape, or other image), and the like. In the example of FIGS. 14A and 14B, the one or more markings 1402 are implemented in the form of a roman numeral I indentation on one side of the tip structure 716 and a roman numeral II indentation on the opposite side of the tip structure 716. In some embodiments, an indentation marking can be filled with a substance to provide a relatively smooth surface on the tip structure 716. In examples, the one or more markings 1402 can be implemented on both the outer diameter and the inner diameter of the tip structure 716, as shown. However, the one or more markings 1402 can be implemented on one edge, such as the inner edge/diameter or the outer edge/diameter. In instances where the one or more markings 1402 are implemented as indentations on only an inner diameter/edge, the tip structure 716 can provide a smooth outer edge, which can be advantageous in some cases to navigate the shaft 604 while avoiding snags on anatomy of a patient. In some embodiments, the one or more markings 1402 are implemented in a particular manner that is more easily detectable by image processing techniques, such as a pattern, image (e.g., QR code), etc. Although the one or more markings 1402 are illustrated on the tip structure 716, the one or more markings 1402 can be located at other locations, such as another portion of the shaft 604.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. may be either X, Y, or Z. Thus, such conjunctive language is not generally intended to imply that certain embodiments require at least one of X, at least one of Y, and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the disclosure herein should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A catheter comprising:
an elongate shaft including a distal section, a middle section, a proximal section, and a lumen, the middle section including a first inner diameter, at least a portion of the distal section including a second inner diameter that is smaller than the first inner diameter, wherein the lumen is configured to couple to an aspiration system to provide aspiration to a target site via the lumen; and
an instrument base coupled to the elongate shaft and configured to control actuation of the elongate shaft.

2. The catheter of claim 1, wherein a ratio of the second inner diameter to the first inner diameter is within a range of 0.5 to 0.9.

3. The catheter of claim 1, wherein a longitudinal length of the at least the portion of the distal section that includes the second inner diameter is less than the second inner diameter.

4. The catheter of claim 1, wherein the distal section of the elongate shaft includes a first portion and a second portion that is distal to the first portion, the first portion including the second inner diameter, the second portion including a third inner diameter that is larger than the second inner diameter.

5. The catheter of claim 4, wherein a longitudinal length of the first portion is less than the second inner diameter.

6. The catheter of claim 4, further comprising:
an elongate movement member slidably disposed in a wall lumen in the elongate shaft, the elongate movement member being coupled to the first portion;
wherein the instrument base is configured to manipulate the elongate movement member to control actuation of the elongate shaft.

7. The catheter of claim 1, wherein the distal section of the elongate shaft is removably coupled to the middle section of the elongate shaft.

8. An aspiration catheter comprising:
an elongate shaft configured to couple to an aspiration system, the elongate shaft including a proximal portion, a medial portion, a tip portion, and a lumen extending from the proximal portion to the tip portion, the medial portion including a first inner diameter that is larger than a second inner diameter of the tip portion, the tip portion being configured to removably receive debris within a patient; and
an instrument handle coupled to the elongate shaft and configured to manipulate the elongate shaft to control actuation of the elongate shaft.

9. The aspiration catheter of claim 8, wherein the tip portion includes at least one of a counterbore or a countersink.

10. The aspiration catheter of claim 8, wherein a length of the tip portion is less than the second inner diameter of the tip portion.

11. The aspiration catheter of claim 8, wherein a ratio of the second inner diameter of the tip portion to the first inner diameter of the medial portion is within a range of 0.5 to 0.9.

12. The aspiration catheter of claim 8, further comprising:
a pull wire slidably disposed in a wire lumen in the elongate shaft, the pull wire being coupled to the tip portion;
wherein the instrument handle is configured to manipulate the pull wire to control actuation of the elongate shaft.

13. The aspiration catheter of claim 8, wherein the tip portion includes a first portion and a second portion that is distal to the first portion, the first portion including the second inner diameter, the second portion including a third inner diameter that is larger than the second inner diameter.

14. The aspiration catheter of claim 8, wherein the tip portion includes one or more orientation markings.

15. A system comprising:
an elongate shaft including a proximal portion, a medial portion, a tip portion, and a first lumen extending from the proximal portion to the tip portion, the medial portion including a first inner diameter that is larger than a second inner diameter of the tip portion, the first lumen being configured to couple to an aspiration system to provide aspiration to a target site via the first lumen; and
an elongate movement member slidably disposed in a second lumen in the elongate shaft, the elongate movement member being coupled to the tip portion and configured to control actuation of the elongate shaft.

16. The system of claim 15, wherein a ratio of the second inner diameter to the first inner diameter is within a range of 0.5 to 0.9.

17. The system of claim 15, wherein the tip portion includes a first section and a second section that is distal to the first section, the first section including the second inner diameter, the second section including a third inner diameter that is larger than the second inner diameter.

18. The system of claim 17, wherein a longitudinal length of the first section is less than the second inner diameter.

19. The system of claim 15, wherein the tip portion has a fracture toughness greater than or equal to 2 MPa·m^{1/2}.

20. The system of claim 15, wherein the tip portion includes at least one of stainless steel, titanium, tungsten, aluminum alloy, iron alloy, steel alloy, titanium alloy, or tungsten alloy.
